(19) United States
(12) Patent Application Publication
(10) Pub. No.: US 2023/0070505 A1
(43) Pub. Date: Mar. 9, 2023

(54) COMPOSITIONS AND METHODS FOR ANALYTE DETECTION

(71) Applicant: Colorado State University Research Foundation, Fort Collins, CO (US)

(72) Inventors: Claudia Gentry-Weeks, Fort Collins, CO (US); Christie Peebles, Fort Collins, CO (US); Kevin Morey, Fort Collins, CO (US)

(21) Appl. No.: 17/929,574

(22) Filed: Sep. 2, 2022

Related U.S. Application Data
(60) Provisional application No. 63/240,426, filed on Sep. 3, 2021.

(51) Int. Cl. C12Q 1/70; G01N 21/64

(52) U.S. Cl. CPC C12Q 1/701; G01N 21/6428; G01N 2021/6432; C12Q 1/6818

(57) ABSTRACT

The present disclosure provides compositions and methods related to the detection of an analyte-of-interest. In particular, the present disclosure provides compositions and methods related to the detection and/or quantification of an analyte-of-interest using a signal detection component in combination with a signal amplification component. By combining these components in a modular format, cell-free synthetic gene circuits can be generated or improved to address a specific biological or biomedical diagnostic need.

### CROSS REFERENCE TO RELATED APPLICATION

This application claims priority to and the benefit of U.S. Provisional Patent Application Ser. No. 63/240,426 filed Sep. 3, 2022, which is incorporated herein by reference in its entirety and for all purposes.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: 62,276 Byte XML file named “CSU-41137-202,” created on Sep. 2, 2022.

### FIELD

The present disclosure provides compositions and methods related to the detection of an analyte-of-interest. In particular, the present disclosure provides compositions and methods related to the detection and/or quantification of an analyte-of-interest using a signal detection component in combination with a signal amplification component. By combining these components in a modular format, cell-free synthetic gene circuits can be generated or improved to address a specific biological or biomedical diagnostic need.

### BACKGROUND

Synthetic biology offers biotechnological solutions to modern medical problems through the use of gene circuits with on/off switches which are expressed in cell free protein expression systems for the transcription and translation of a diagnostic end product. For example, the addition of a riboswitch in a gene circuit serves as an on/off switch since the resulting RNA has secondary structure, self-binding, and the ability to regulate the translational expression of a gene encoded on the same mRNA strand. Upon interaction with an inducer molecule, the riboswitch will alter its structure to allow translation of a functional protein, often a reporter gene. By combining these components in a modular format, cell-free synthetic gene circuits can be easily fine-tuned to meet a specific biological or medical diagnostic need. Additionally, cell-free synthetic gene circuits have recently been developed for the detection of viral RNA particles in point-of-care devices. Although recent world events have seen new interest in developing rapid and inexpensive medical diagnostic platforms, significant obstacles remain, including the requirement for large concentrations of inducer molecules (e.g., analytes-of-interest) for obtaining a successful detection.

### SUMMARY

Embodiments of the present disclosure include a composition for performing an analyte detection assay. In accordance with these embodiments, the composition includes a detection component comprising an RNA molecule capable of binding an analyte, wherein the RNA molecule comprises an analyte recognition domain, a ribosome binding site, and an initiator protein domain; and a signal amplification component responsive to expression of an initiator protein encoded by the initiator protein domain. In some embodiments, the presence of the analyte causes expression of the initiator protein and subsequent activation of the signal amplification component.

In some embodiments, the RNA molecule forms a hairpin structure, wherein the hairpin structure is disrupted upon binding of the analyte to the analyte recognition domain, thereby exposing ribosome binding site and the initiator protein domain.

In some embodiments, the initiator protein comprises an enzyme. In some embodiments, the enzyme is adenylate cyclase, or a derivative or variant thereof. In some embodiments, the adenylate cyclase, or a derivative or variant thereof, is from a bacterium, a virus, an archaeon, a fungus, a protozoan, a vertebrate, a non-vertebrate, or a plant. In some embodiments, the adenylate cyclase, or a derivative or variant thereof, is from Bordetella pertussis.

In some embodiments, the enzyme is a protease, or a derivative or variant thereof. In some embodiments, the protease, or a derivative or variant thereof, is from a bacterium, a virus, an archaeon, a fungus, a protozoan, a vertebrate, a non-vertebrate, or a plant. In some embodiments, the protease, or a derivative or variant thereof, is a plant virus protease. In some embodiments, the plant virus protease is a tobacco etch virus (TEV) protease, a papain-like cystine protease, or a glutamic protease.

In some embodiments, the signal amplification component includes one or more of the following: (i) a peptide comprising a protease cleavage site coupled to a fluorescent molecule and a quencher molecule, wherein the fluorescent molecule is activated upon cleavage of the protease cleavage cite by a protease and/or a cascade of proteases; (ii) a luciferase complex comprising luciferin, or a derivative or variant thereof, and a luciferase or split luciferase comprising a protease cleavage site, wherein the luciferase or split luciferase is activated upon cleavage of the protease cleavage site by a protease and/or a cascade of proteases; (iii) a fluorescent protein or polypeptide comprising a protease cleavage site, wherein the fluorescent protein or polypeptide is activated upon cleavage of the protease cleavage site by a protease and/or a cascade of proteases; (iv) one or more components of a glycogenolysis complex; (v) one or more components of a beta-galactosidase complex; or (vi) one or more nucleic acid aptamers.

In some embodiments, the peptide comprises a TEV-specific protease cleavage site comprising ENLYFQG (SEQ ID NO: 42), wherein the fluorescent molecule is a 5-FAM dye, and wherein the quencher is a QLX 520 quencher. In some embodiments, the one or more components of a glycogenolysis complex comprises: (i) Protein Kinase A, Phosphorylase Kinase b, and Glycogen Phosphorylase, or phosphorylated derivatives thereof; and (ii) cAMP, ADP, and glucose-1-P. In some embodiments, the fluorescent protein or polypeptide comprises GFP, mNeonGreen, YFP, RFP, or CFP, or a variant or derivative thereof. In some embodiments, the one or more components of a beta-galactosidase complex comprises: (i) 5-bromo-4-chloro-3-indolyl-β-D-galactopyranoside (X-gal), ortho-Nitrophenyl-ß-galactoside (ONPG), 6-chloro-3-indolyl-β-D-galactopyranoside (S-gal), 5-bromo-6-chloro-3-indolyl-β-D-galactopyranoside (Magenta-gal), 5-bromo-3-indolyl-β-D-galactopyranoside (Bluo-gal), p-nitrophenyl-β-d-galactopyranoside (PNPG); and (ii) beta-galactosidase, or a derivative or variant thereof. In some embodiments, the one or more nucleic acid aptamers comprises a Broccoli 3WJdB aptamer, and wherein the signal amplification component further comprises a DFHBI-1T dye.

In some embodiments, the composition comprises one or more components of a cell free protein expression system. In some embodiments, the composition comprises a reaction buffer.

In some embodiments, the analyte comprises at least one of a DNA molecule, an RNA molecule, a small molecule, a lipid, a peptide, a polypeptide, a protein, or a glycoprotein. In some embodiments, the analyte is from a pathogenic organism selected from the group consisting of bacteria, viruses, protozoa, worms, and fungi. In some embodiments, the analyte comprises RNA from a virus. In some embodiments, the virus is a SARS-CoV-2.

In some embodiments, the RNA molecule comprises the nucleic acid sequence: GGGNNNNNNNNNNNNNNNNNNNNNNNNNNNNNNAACAGAGGAGANNNNNNAU GNNNNNNNNNAACGGUAGCGCAGGUAGCGGCAUAUG (SEQ ID NO: 1). In some embodiments, the RNA molecule comprises a nucleic acid sequence selected from the group consisting of SEQ ID NOs: 2-19.

Embodiments of the present disclosure also include a method for detecting an analyte that includes combining a composition comprising any of the detection components and any of the signal amplification components described herein with a biological sample; and measuring or detecting a signal produced by the signal amplification component. In some embodiments, the presence of the analyte in the biological sample causes expression of an initiator protein and subsequent activation of the signal amplification component.

In some embodiments, the biological sample comprises a blood sample, a plasma sample, a serum sample, a cerebral spinal fluid sample, a saliva sample, a tear sample, a urine sample, a fecal sample, a cell sample, a tissue sample, a water sample, and/or a plant sample.

In some embodiments, the signal is a fluorescent signal, a bioluminescent signal, a chemical signal, an electrochemical signal, or a colorimetric signal.

In some embodiments, the method further comprises quantifying the signal and determining a concentration of the analyte in the biological sample. In some embodiments, the analyte is present in the sample at a concentration ranging from about 1 fM to about 1 μM In some embodiments, the analyte comprises viral RNA from SARS-CoV-2.

Embodiments of the present disclosure also include a kit comprising any of the compositions described herein and instructions for performing an analyte detection assay. In some embodiments, the kit comprises one or more components of a cell free protein expression system and/or a reaction buffer.

Embodiments of the present disclosure also include a composition for performing an analyte detection assay. In accordance with these embodiments, the composition includes a detection component comprising an allosteric transcription factor comprising an analyte recognition domain, and a DNA molecule comprising an allosteric transcription factor binding domain, a ribosome binding site, and an initiator protein domain; and a signal amplification component responsive to expression of an initiator protein encoded by the initiator protein domain. In some embodiments, the presence of the analyte causes expression of the initiator protein and subsequent activation of the signal amplification component.

In some embodiments, the allosteric transcription factor is selected from the group consisting of: (i) a TetR transcription factor selected from TetR, MphR, QacR, and TtgR, and any variants or derivates thereof; (ii) a multiple antibiotic resistance repressor (MarR) transcription factors selected from OtrR, CtcS, MobR, and HucR, and any variants or derivates thereof; (iii) an ArsR/SmtB transcriptional regulator selected from SmtB and CadC, and any variants or derivates thereof; (iv) a CsoR/RcnR transcriptional regulator selected from CsoR, and any variants or derivates thereof; (v) a MerR transcriptional regulator, and any variants or derivates thereof; (vi) a Fur transcriptional regulator, and any variants or derivates thereof; (vii) a DtxR transcriptional regulator, and any variants or derivates thereof; (viii) a NikR transcriptional regulator, and any variants or derivates thereof; and (ix) a xylR transcriptional activator acting on toluene, xylene, benzene, and any variants or derivatives thereof.

In some embodiments, the initiator protein comprises an enzyme. In some embodiments, the enzyme is adenylate cyclase, or a derivative or variant thereof. In some embodiments, the adenylate cyclase, or a derivative or variant thereof, is from a bacterium, a virus, an archaeon, a fungus, a protozoan, a vertebrate, a non-vertebrate, or a plant. In some embodiments, the adenylate cyclase, or a derivative or variant thereof, is from Bordetella pertussis.

In some embodiments, the enzyme is a protease, or a derivative or variant thereof. In some embodiments, the protease, or a derivative or variant thereof, is from a bacterium, a virus, an archaeon, a fungus, a protozoan, a vertebrate, a non-vertebrate, or a plant. In some embodiments, the protease, or a derivative or variant thereof, is a plant virus protease. In some embodiments, the plant virus protease is a tobacco etch virus (TEV) protease, a papain-like cystine protease, or a glutamic protease.

In some embodiments, the signal amplification component includes one or more of the following: (i) a peptide comprising a protease cleavage site coupled to a fluorescent molecule and a quencher molecule, wherein the fluorescent molecule is activated upon cleavage of the protease cleavage cite by a protease and/or a cascade of proteases; (ii) a luciferase complex comprising luciferin, or a derivative or variant thereof, and a luciferase or split luciferase comprising a protease cleavage site, wherein the luciferase or split luciferase is activated upon cleavage of the protease cleavage site by a protease and/or a cascade of proteases; (iii) a fluorescent protein or polypeptide comprising a protease cleavage site, wherein the fluorescent protein or polypeptide is activated upon cleavage of the protease cleavage site by a protease and/or a cascade of proteases; (iv) one or more components of a glycogenolysis complex; (v) one or more components of a beta-galactosidase complex; or (vi) one or more nucleic acid aptamers.

In some embodiments, the peptide comprises a TEV-specific protease cleavage site comprising ENLYFQG (SEQ ID NO: 42), wherein the fluorescent molecule is a 5-FAM dye, and wherein the quencher is a QLX 520 quencher.

In some embodiments, the one or more components of a glycogenolysis complex comprises: (i) Protein Kinase A, Phosphorylase Kinase b, and Glycogen Phosphorylase, or phosphorylated derivatives thereof; and (ii) cAMP, ADP, and glucose-1-P. In some embodiments, the fluorescent protein or polypeptide comprises GFP, mNeonGreen, YFP, RFP, or CFP, or a variant or derivative thereof. In some embodiments, the one or more components of a beta-galactosidase complex comprises: (i) 5-bromo-4-chloro-3-indolyl-β-D-galactopyranoside (X-gal), ortho-Nitrophenyl-ß-galactoside (ONPG), 6-chloro-3-indolyl-β-D-galactopyranoside (S-gal), 5-bromo-6-chloro-3-indolyl-β-D-galactopyranoside (Magenta-gal), 5-bromo-3-indolyl-β-D-galactopyranoside (Bluo-gal), p-nitrophenyl-β-d-galactopyranoside (PNPG); and (ii) beta-galactosidase, or a derivative or variant thereof. In some embodiments, the one or more nucleic acid aptamers comprises a Broccoli 3WJdB aptamer, and wherein the signal amplification component further comprises a DFHBI-1T dye.

In some embodiments, the composition comprises one or more components of a cell free protein expression system. In some embodiments, the composition comprises a reaction buffer.

In some embodiments, the analyte comprises at least one of a DNA molecule, an RNA molecule, a small molecule, a lipid, a peptide, a polypeptide, a protein, or a glycoprotein.

Embodiments of the present disclosure also include a method for detecting an analyte. In accordance with these embodiments, the method includes combining a composition comprising any of the detection components and any of the signal amplification components described herein with a biological sample; and measuring or detecting a signal produced by the signal amplification component. In some embodiments, the presence of the analyte in the biological sample causes expression of an initiator protein and subsequent activation of the signal amplification component.

In some embodiments, the biological sample comprises a blood sample, a plasma sample, a serum sample, a cerebral spinal fluid sample, a saliva sample, a tear sample, a urine sample, a fecal sample, a cell sample, a tissue sample, a water sample, and/or a plant sample.

In some embodiments, the signal is a fluorescent signal, a bioluminescent signal, a chemical signal, an electrochemical signal, or a colorimetric signal.

In some embodiments, the method further comprises quantifying the signal and determining a concentration of the analyte in the biological sample. In some embodiments, the analyte is present in the sample at a concentration ranging from about 1 fM to about 1 μM.

### BRIEF DESCRIPTION OF THE DRAWINGS

FIGS. 1A-1B: Schematic illustration of two embodiments of the analyte detection systems of the present disclosure (FIG. 1A). Scheme 1 includes an RNA-based detection component (e.g., toehold switch), and Scheme 2 includes a detection component that uses an allosteric transcription factor domain. Additional representative schematic illustration of the various uses of the composition and methods of the present disclosure as part of a platform for detecting a variety of different analytes (FIG. 1B).

FIGS. 2A-2B: Schematic illustration of signal amplification components of the present disclosure, which can include the use of a single protease or a multi-protease cascade (FIG. 2A). Additional schematic illustration of one embodiment of a signal amplification component of the present disclosure, which includes a glycogenolysis multienzyme cascade (FIG. 2B).

FIG. 3: Representative consensus RNA sequence for toehold switches described in the present disclosure, including a linker region operably coupled to a reporter protein.

FIG. 4: Construction of the pUC19-T7 expression plasmids containing toehold switches upstream of the mNeonGreen reporter gene. A similar process was used for cloning RNA inducer sequences in the pUC19-T7 expression plasmid.

FIG. 5: Schematic illustration of the location of toehold switches to their respective locations on the SARS-CoV-2 genome (from nt 1,865 to 2,018). Region 1 corresponds to nt 1918-1933 and region 2 corresponds to nt 1865-2018.

FIG. 6: Representative fold change plots showing the initial screening of toehold sensors and inducers. Error bars represent +/−1 Standard Deviation from the mean.

FIG. 7: Representative data that includes a limit of detection dilution series using the mNeonGreen reporter protein with 115 nt and 5,100 nt SARS-CoV-2-derived RNA inducer. Error bars represent +/−1 standard deviation from the mean.

FIG. 8: Representative data that includes a limit of detection dilution series using the 135 nt and the 5,100 nt SARS-CoV-2-derived RNA inducer with the TEV Protease reporter amplification system. Error bars represent +/−1 Standard Deviation from the mean.

FIG. 9: Representative data from cell free protein synthesis reporter experiments using mNeonGreen and eGFP. About 125 ng of reporter gene plasmid added to 25 ml NEBExpress Cell-free reaction. Negative control was a reaction with a non-fluorescent protein. Reactions incubated at 37° C. for 1 hour. Reactions transferred to 96 well plate. Fluorescent plate reader assay: 485 nm excitation 520 nm emission.

FIG. 10: Representative data demonstrating the specificity of the assays of the present disclosure, including for the detection of large nucleic acid targets.

### DETAILED DESCRIPTION

As evidenced by the SARS-CoV-2 and MERS-CoV outbreaks, diseases caused by RNA viruses can quickly become global pandemics. As a result, an increased emphasis has been placed on applying synthetic biology techniques toward rapid detection of pathogenic organisms, such as RNA viruses. The ideal diagnostic test should be inexpensive, lack the need for lengthy antibody production or PCR amplification, and be modular in nature so it can be easily and rapidly adapted to new and emerging threats. Current diagnostic methods rely on technical expertise, costly equipment, and often either utilize monoclonal antibodies for capture or detection or use RT-PCR to amplify the viral RNA, which can limit the accessibility of these diagnostics to the public. A combination of a modular synthetic gene circuit with a switch control mechanism, a downstream enzymatic signal amplification system, and cell-free protein synthesis offers the ability to perform rapid and sensitive detection diagnostics without the need for expensive equipment and can be adapted for distribution and storage on an inexpensive substrate such as paper.

Previous work established that riboswitches form a stem-loop structure (referred to as a “toehold switch” due to its shape) with an inaccessible AUG start codon and serve as an on/off switch for expression of a downstream green fluorescent protein (GFP) reporter gene. Binding of a target nucleic acid sequence (e.g., the “inducer” or “trigger’) to the toehold causes strand invasion of the stem loop structure, resulting in disruption of the stem loop structure which allows translation and synthesis of a detectable GFP. In studies by Pardee et al., a Loop-Mediated Isothermal Amplification (LAMP) step was employed prior to the detection process to increase the amount of viral RNA inducer available for binding the toehold switch. They first amplified Zika viral RNA in the sample and incorporated a downstream β-galactosidase colorimetric signal for detection. Similar strategies have been used to design toehold switches that bind SARS-CoV-2 viral RNA, resulting in translation of a detectable signal with a limit of detection (LOD) of 30 nM.

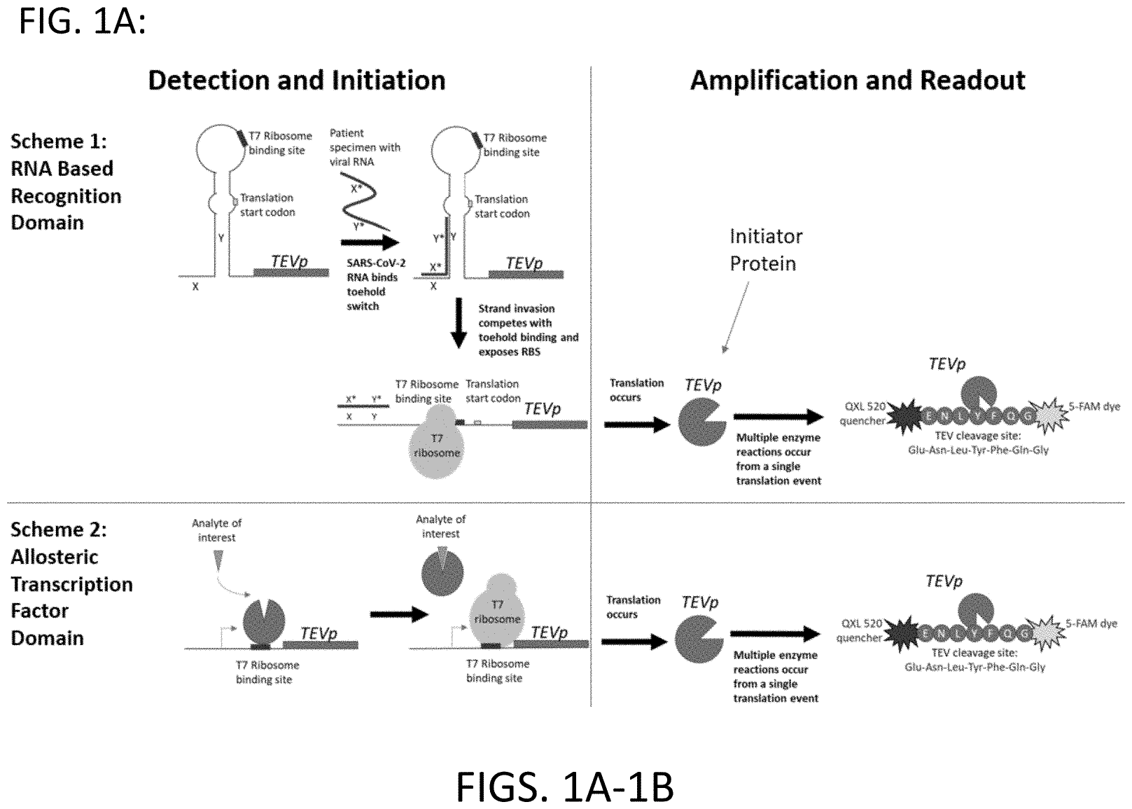

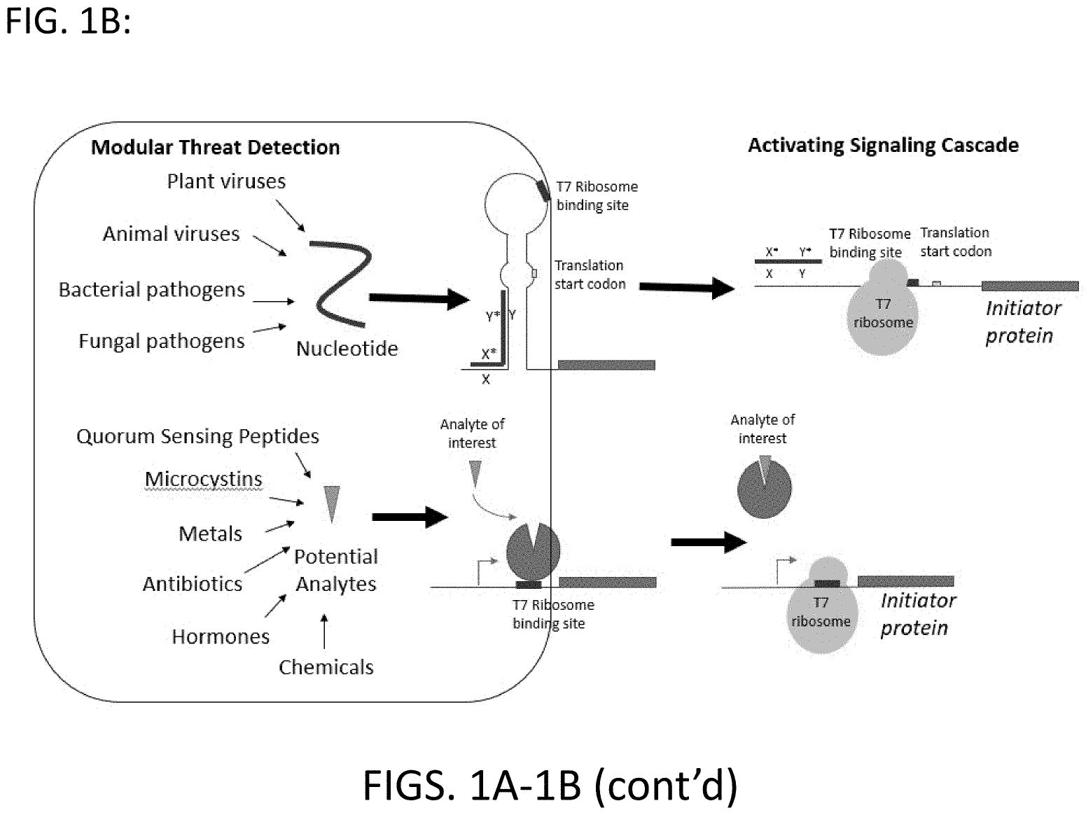

Embodiments of the present disclosure provide enhanced compositions and methods for improving SARS-CoV-2 detection, including a downstream amplification system (FIG. 1) using a tobacco mosaic etch virus (TEV) protease detection system; use of improved detection readouts, such as the use of mNeonGreen, a more stable and intense derivative of the yellow fluorescent protein derived from Branchiostoma lancelotum; and selective design of a toehold switch to avoid both interfering secondary structure in the viral RNA inducer region and to avoid regions in the viral genome that are more likely to undergo spontaneous mutations.

For toehold switch design, rather than scan the entire SARS-CoV-2 viral genome and generate and test toehold switches, a focused approach was used to identify regions for optimal and specific binding of viral RNA to the toehold switch. First, a region of the viral genome for toehold design was chosen that was conserved among SARS-CoV-2 viruses but not conserved among human coronavirus strains that cause mild respiratory disease, such as HCoV-OC43, HCoV-229E, and HCoV-NL6 (cdc.gov/coronavirus/types.html). Second, the toehold binding region was narrowed down to avoid the pseudoknot between orfA and orfB genes, untranslated regions (UTRs), regions with local secondary structure and regions encoding the spike protein since this would naturally be more prone to mutations. Third, the toehold binding region was intentionally designed to avoid the structural protein genes that would be susceptible to mutation due to selective pressure from vaccination or infection. The rationale for this combined, focused approach was to generate toehold switches that would be both sensitive and selective for SARS-CoV-2, would more likely remain functional even with the expected mutations associated with an RNA virus, and be relatively free of secondary structure that might otherwise inhibit binding to the toehold switch.

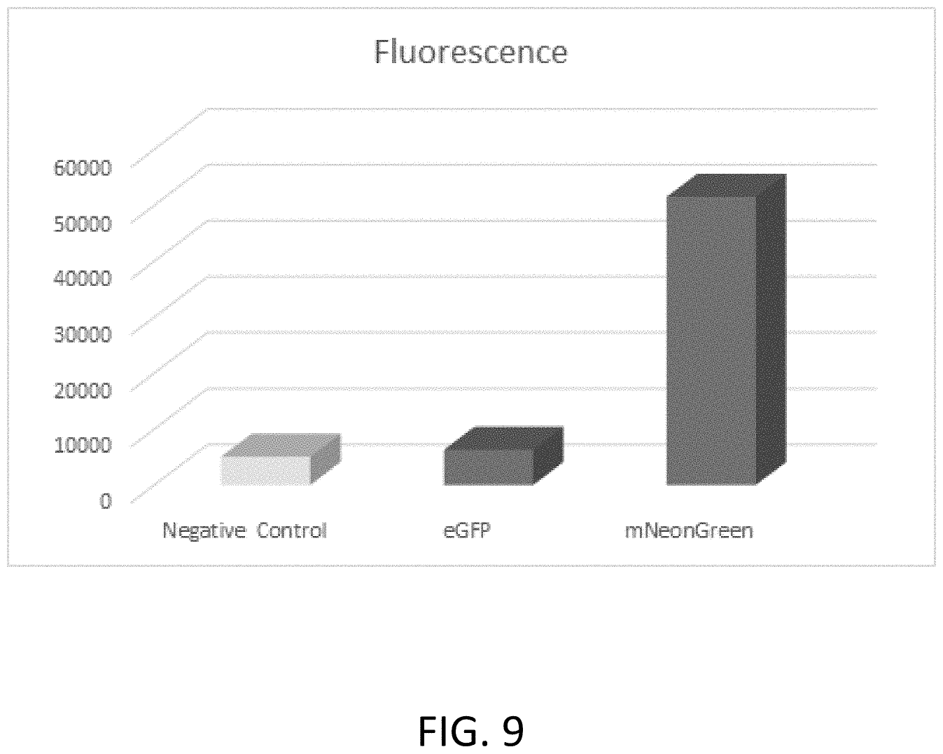

Rather than use upstream amplification of the viral RNA, experiments focused on amplification of the downstream detection signal to avoid the pitfalls associated with amplification of SARS-CoV-2 viral RNA from complicated substrates such as saliva or nasopharyngeal swabs. For proof of concept, mNeonGreen was initially used as the detection signal since this fluorescent protein is three times more intense than GFP and thus should allow more sensitive detection following binding of the toehold switch to the viral RNA. Experiments were conducted to compare intensity of mNeonGreen to eGFP generated in the cell free system, and after 1 hour of synthesis at 37° C., the mNeonGreen sample was ca. 5 times as intense (FIG. 9). Toehold switches were identified that bound to SARS-CoV-2 genomic inducer RNA, allowing subsequent stem loop relaxation and translation of the reporter gene. Once toehold switches were identified that successfully produced a positive signal using the mNeonGreen reporter, a new downstream amplification system based on TEV protease was engineered and tested for increased sensitivity.

Thus, embodiments of the present disclosure describe compositions and methods for detecting an analyte-of-interest that integrate downstream novel amplification components (e.g., using a TEV protease cleavage of a quenched fluorescent reporter) with an enhanced detection component that provides the increased sensitivity of synthetic gene circuit biology-based sensors (e.g., toehold switches and allosteric enzymes). As described further herein, TEV protease is a highly sequence-specific cysteine protease from the Tobacco Etch Virus. Due to its high sequence specificity, it is frequently used for the controlled cleavage of fusion proteins in vitro and in vivo. The SARS-CoV-2 detection system described here uses toehold switches for specific binding of the target pathogen nucleic acid, followed by the in vitro translation of TEV protease. The TEV protease amplifies the readout signal by cleaving a unique site in multiple quenched substrate molecules to provide an enhanced fluorescent signal. The advantages of this amplification system are that the reactions can be run at a single temperature (4° C. to 42° C.) so a thermocycler is not required, nucleic acid amplification is not needed so extensive primer design and testing are not required, and added exogenous enzymes are not essential for the reaction reducing the cost and increasing the stability of the assay. In addition, with this downstream amplification strategy, alternate enzymes can be employed for sensitive colorimetric detection and multiplexing of the diagnostic assay

The use of toehold switches with upstream RNA amplification has been successful, reaching a limit of detection in the low, clinically relevant femtomolar level. However, point-of-care or field diagnostic assays demand an easy to use, inexpensive format that can withstand harsh environmental conditions and has the ability to multiplex assays for differential diagnosis. Although RT-qPCR, NASBA (nucleic acid sequence-based amplification) and RT-LAMP can provide sensitive and specific detection of viruses, they each have different characteristics that may be challenging for use as low-cost, point-of-care detection assays. RT-qPCR requires the use of expensive equipment and technical expertise for precision handling of the reagents and matrix inhibitors can interfere with nucleic acid amplification. NASBA requires three enzymes (e.g., reverse transcriptase, RNAse H, and DNA polymerase) for transcribing single stranded RNA into cDNA, removal of template RNA, and production of double-stranded DNA that stimulates a signal, respectively. RT-LAMP (reverse transcription loop-mediated amplification) is tolerant to matrix inhibitors; however, it requires 4-6 primers that must be carefully designed and extensively tested prior to use. In addition, RT-LAMP is not amenable to multiplexing. Embodiments of the present disclosure were able to reach a clinically relevant level of detection without the need for upstream RNA amplification.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The term “derived from” as used herein refers to cells or a biological sample (e.g., blood, tissue, bodily fluids, plants, etc.) and indicates that the cells or the biological sample were obtained from the stated source at some point in time. For example, a cell derived from an individual can represent a primary cell obtained directly from the individual (e.g., unmodified). In some instances, a cell derived from a given source undergoes one or more rounds of cell division and/or cell differentiation such that the original cell no longer exists, but the continuing cell (e.g., daughter cells from all generations) will be understood to be derived from the same source. The term includes directly obtained from, isolated and cultured, or obtained, frozen, and thawed. The term “derived from” may also refer to a component or fragment of a cell obtained from a tissue or cell, including, but not limited to, a protein, a nucleic acid, a membrane or fragment of a membrane, and the like.

The term “isolating” or “isolated” when referring to a cell or a molecule (e.g., nucleic acids or protein) indicates that the cell or molecule is or has been separated from its natural, original or previous environment. For example, an isolated cell can be removed from a tissue derived from its host individual, but can exist in the presence of other cells (e.g., in culture), or be reintroduced into its host individual.

“Subject” and “patient” as used herein interchangeably refers to any vertebrate, including, but not limited to, a mammal and a human. In some embodiments, the subject may be a human or a non-human. The subject or patient may be undergoing other forms of treatment.

“Mammal” as used herein refers to any member of the class Mammalia, including, without limitation, humans and nonhuman primates such as chimpanzees and other apes and monkey species; farm animals such as cattle, sheep, pigs, goats, llamas, camels, and horses; domestic mammals such as dogs and cats; laboratory animals including rodents such as mice, rats, rabbits, guinea pigs, and the like. The term does not denote a particular age or sex. Thus, adult and newborn subjects, as well as fetuses, whether male or female, are intended to be included within the scope of this term.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease (e.g., viral infection). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

“Coefficient of variation” (CV), also known as “relative variability,” is equal to the standard deviation of a distribution divided by its mean.

“Controls” as used herein generally refers to a reagent whose purpose is to evaluate the performance of a measurement system in order to assure that it continues to produce results within permissible boundaries (e.g., boundaries ranging from measures appropriate for a research use assay on one end to analytic boundaries established by quality specifications for a commercial assay on the other end). To accomplish this, a control should be indicative of patient results and optionally should somehow assess the impact of error on the measurement (e.g., error due to reagent stability, calibrator variability, instrument variability, and the like).

“Dynamic range” as used herein refers to range over which an assay readout is proportional to the amount of target molecule or analyte in the sample being analyzed. The dynamic range can be the range of linearity of the standard curve.

“Limit of Blank (LoB)” as used herein refers to the highest apparent analyte concentration expected to be found when replicates of a blank sample containing no analyte are tested.

“Limit of Detection (LoD)” as used herein refers to the lowest concentration of the measurand (i.e. a quantity intended to be measured) that can be detected at a specified level of confidence. The level of confidence is typically 95%, with a 5% likelihood of a false negative measurement. LoD is the lowest analyte concentration likely to be reliably distinguished from the LoB and at which detection is feasible. LoD can be determined by utilizing both the measured LoB and test replicates of a sample known to contain a low concentration of analyte. The LoD term used herein is based on the definition from Clinical and Laboratory Standards Institute (CLSI) protocol EP17-A2 (“Protocols for Determination of Limits of Detection and Limits of Quantitation; Approved Guideline—Second Edition,” EP17A2E, by James F. Pierson-Perry et al., Clinical and Laboratory Standards Institute, Jun. 1, 2012).

“Limit of Quantitation (LoQ)” as used herein refers to the lowest concentration at which the analyte can not only be reliably detected but at which some predefined goals for bias and imprecision are met. The LoQ may be equivalent to the LoD or it could be at a much higher concentration.

“Linearity” refers to how well the method or assay's actual performance across a specified operating range approximates a straight line. Linearity can be measured in terms of a deviation, or non-linearity, from an ideal straight line. “Deviations from linearity” can be expressed in terms of percent of full scale. In some of the methods disclosed herein, less than 10% deviation from linearity (DL) is achieved over the dynamic range of the assay. “Linear” means that there is less than or equal to about 20%, about 19%, about 18%, about 17%, about 16%, about 15%, about 14%, about 13%, about 12%, about 11%, about 10%, about 9%, or about 8% variation for or over an exemplary range or value recited.

“Sensitivity” as used herein generally refers to the percentage of true positives that are predicted by a test to be positive, while “specificity,” as used herein refers to the percentage of true negatives that are predicted by a test to be negative. For example, a ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

“Reference level” as used herein refers to an assay cutoff value that is used to assess diagnostic, prognostic, or therapeutic efficacy and that has been linked or is associated herein with various clinical parameters (e.g., presence of disease, stage of disease, severity of disease, progression, non-progression, or improvement of disease, etc.). However, it is well-known that reference levels may vary depending on the nature of the assay used and that assays can be compared and standardized. It further is well within the ordinary skill of one in the art to adapt the disclosure herein for other assays to obtain specific reference levels for those other assays based on the description provided by this disclosure. Whereas the precise value of the reference level may vary between assays, the findings as described herein should be generally applicable and capable of being extrapolated to other assays.

“Sample,” “test sample,” “specimen,” “sample from a subject,” and “patient sample” as used herein may be used interchangeably and may be a sample of blood, such as whole blood, tissue, skin, urine, serum, plasma, saliva, amniotic fluid, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

### 2. ANALYTE DETECTION ASSAYS

In accordance with the data described herein, embodiments of the present disclosure include a composition for performing an analyte detection assay. In some embodiments, the composition includes a detection component comprising an RNA molecule capable of binding an analyte. The RNA molecule can include an analyte recognition domain, a ribosome binding site, and an initiator protein domain. In some embodiments, the RNA molecule includes at least one additional domain that can augment analyte recognition, ribosome recognition, and/or protein expression. The composition also includes a signal amplification component that is responsive to the expression of an initiator protein encoded by the initiator protein domain. That is, in some embodiments, the presence of the analyte causes expression of the initiator protein and subsequent activation of the signal amplification component. It is in this general manner that the analyte detection component and the signal amplification component are integrated into a function analyte detection system (FIG. 1, upper panels).

Embodiments of the present disclosure also include a composition for performing an analyte detection assay in which the detection component includes an allosteric transcription factor. The allosteric transcription factor includes an analyte recognition domain, as well as domains that facilitate its binding to a portion of an RNA molecule. The composition also includes an RNA molecule comprising an allosteric transcription factor binding domain, a ribosome binding site, and an initiator protein domain. In some embodiments, the RNA molecule includes at least one additional domain that can augment analyte recognition, ribosome recognition, and/or protein expression. This embodiment of the composition also includes a signal amplification component that is responsive to the expression of an initiator protein encoded by the initiator protein domain. That is, in some embodiments, the presence of the analyte causes expression of the initiator protein and subsequent activation of the signal amplification component. It is in this general manner that the analyte detection component and the signal amplification component are integrated into a function analyte detection system (FIG. 1, lower panels).

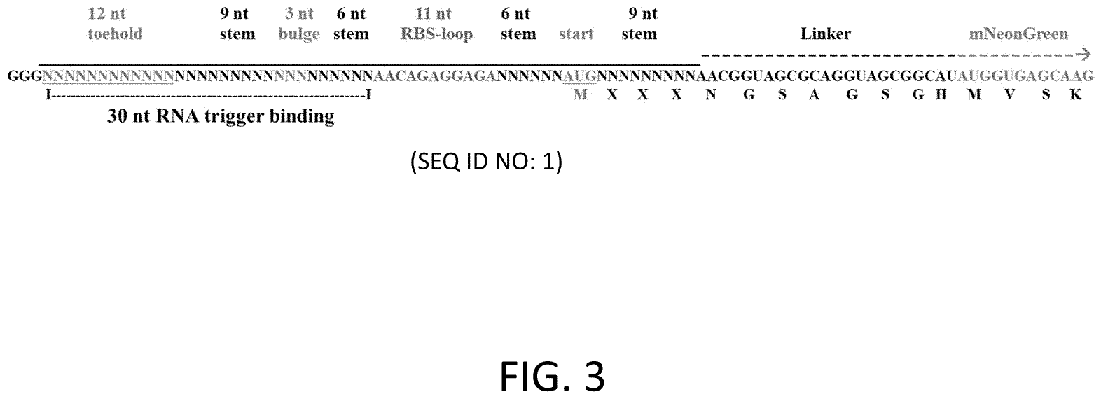

In some embodiments, the RNA molecule capable of binding an analyte can form a hairpin structure (FIG. 3). The hairpin structure can be disrupted upon binding of the analyte (e.g., a polynucleotide) to the analyte recognition domain. In some embodiments, binding of the analyte to the RNA molecule exposes the ribosome binding site and the initiator protein domain on the RNA molecule such that a ribosome can bind and initiate protein synthesis.

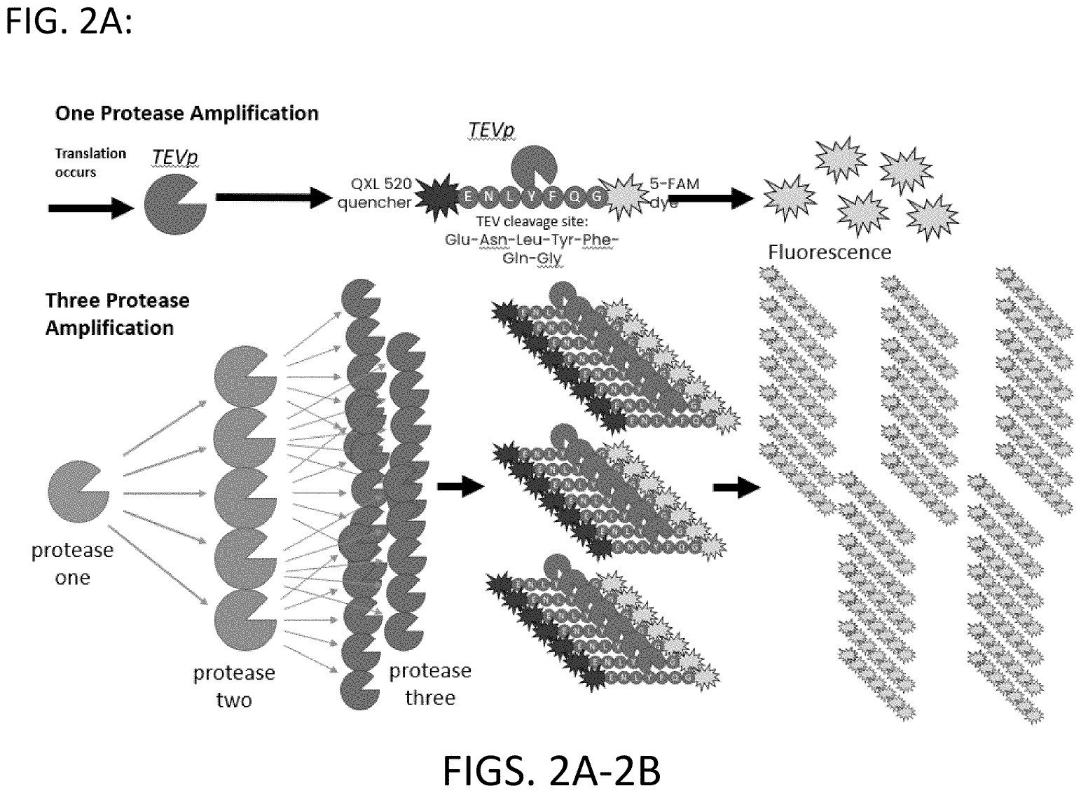

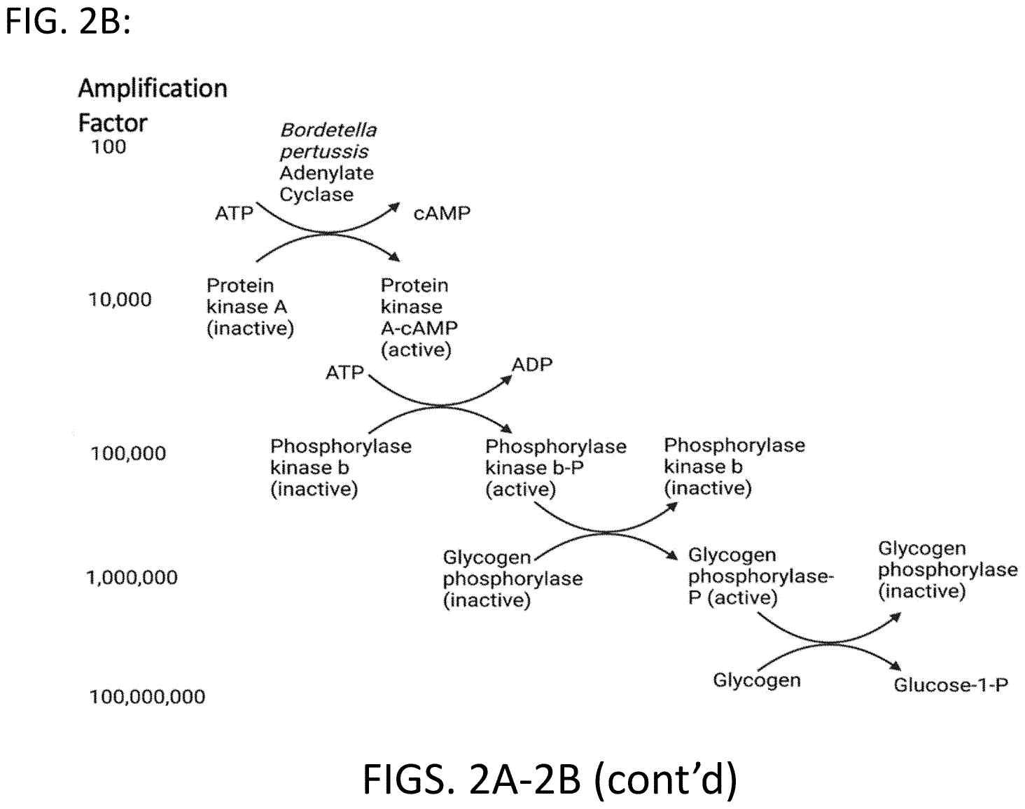

In some embodiments, the initiator protein is an enzyme, or a functional fragment or derivative thereof. In some embodiments, the enzyme is a protease, or a derivative or variant thereof. In some embodiments, the protease, or a derivative or variant thereof, is from a bacterium, a virus, an archaeon, a fungus, a protozoan, a vertebrate, a non-vertebrate, or a plant. In some embodiments, the protease, or a derivative or variant thereof, is a plant virus protease. In some embodiments, the plant virus protease is a tobacco etch virus (TEV) protease, a papain-like cystine protease, or a glutamic protease. In other embodiments, the enzyme is adenylate cyclase, or a derivative or variant thereof. In some embodiments, the adenylate cyclase, or a derivative or variant thereof, is from a bacterium, a virus, an archaeon, a fungus, a protozoan, a vertebrate, a non-vertebrate, or a plant. In some embodiments, the adenylate cyclase, or a derivative or variant thereof, is from Bordetella pertussis (FIG. 2).

Turning to the signal amplification component, in some embodiments, the signal amplification component includes one or more of the following: (i) a peptide comprising a protease cleavage site coupled to a fluorescent molecule and a quencher molecule, wherein the fluorescent molecule is activated upon cleavage of the protease cleavage cite by a protease and/or a cascade of proteases; (ii) a luciferase complex comprising luciferin, or a derivative or variant thereof, and a luciferase or split luciferase comprising a protease cleavage site, wherein the luciferase or split luciferase is activated upon cleavage of the protease cleavage site by a protease and/or a cascade of proteases; (iii) a fluorescent protein or polypeptide comprising a protease cleavage site, wherein the fluorescent protein or polypeptide is activated upon cleavage of the protease cleavage site by a protease and/or a cascade of proteases; (iv) one or more components of a glycogenolysis complex; (v) one or more components of a beta-galactosidase complex; or (vi) one or more nucleic acid aptamers. With any of these embodiments, the signal amplification component is responsive to the initiator protein and facilitates signal amplification such that signal detected corresponds to the quantity of the analyte being detected.

In particular embodiments, the peptide comprising a protease cleavage site coupled to a fluorescent molecule and a quencher molecule comprises a TEV-specific protease cleavage site comprising ENLYFQG (SEQ ID NO: 42). In some embodiments, the fluorescent molecule is a 5-FAM dye, and the quencher is a QLX 520 quencher. In other embodiments, the luciferase complex comprising luciferin, or a derivative or variant thereof, and a luciferase or split luciferase comprising a protease cleavage site is adapted from one or more embodiments described in U.S. Pat. Nos. 8,809,529; 9,797,889; 9,797,890; all of which are incorporated herein by reference in their entireties.

In another embodiment, the one or more components of a glycogenolysis complex comprises: (i) Protein Kinase A, Phosphorylase Kinase b, and Glycogen Phosphorylase, or phosphorylated derivatives thereof; and (ii) cAMP, ADP, and glucose-1-P. In some embodiments, the fluorescent protein or polypeptide comprises GFP, mNeonGreen, YFP, RFP, or CFP, or a variant or derivative thereof. Any other fluorescent or luminescent protein or polypeptide can also be used, as would be recognized by one of ordinary skill in the art based on the present disclosure.

In another embodiment, the one or more components of a beta-galactosidase complex comprises: (i) 5-bromo-4-chloro-3-indolyl-β-D-galactopyranoside (X-gal), ortho-Nitrophenyl-ß-galactoside (ONPG), 6-chloro-3-indolyl-β-D-galactopyranoside (S-gal), 5-bromo-6-chloro-3-indolyl-β-D-galactopyranoside (Magenta-gal), 5-bromo-3-indolyl-β-D-galactopyranoside (Bluo-gal), p-nitrophenyl-β-d-galactopyranoside (PNPG); and (ii) beta-galactosidase, or a derivative or variant thereof.

In another embodiments, the one or more nucleic acid aptamers comprises a Broccoli 3WJdB aptamer. In accordance with these embodiments, the signal amplification component further comprises a DFHBI-1T dye that is responsive to the aptamer. Any dye/aptamer combination also be used, as would be recognized by one of ordinary skill in the art based on the present disclosure.

In accordance with the above embodiments, the compositions of the present disclosure can include one or more components of a cell free protein expression system. In order to express the biologically active proteins of interest described herein (e.g., initiator protein), a cell free protein synthesis system can be used. Cell extracts have been developed that support the synthesis of proteins in vitro from purified mRNA transcripts or from mRNA transcribed from DNA during the in vitro synthesis reaction. Cell free protein synthesis of polypeptides in a reaction mix can comprise cell extracts and/or defined reagents (e.g., buffers). The reaction mix can include at least ATP or an energy source; a template for production of the macromolecule, e.g., DNA, mRNA, etc.; amino acids, and such co-factors, enzymes and other reagents that are necessary for polypeptide synthesis, e.g., ribosomes, tRNA, polymerases, transcriptional factors, aminoacyl synthetases, elongation factors, initiation factors, etc. In one embodiment of the invention, the energy source is a homeostatic energy source. Also included may be enzyme(s) that catalyze the regeneration of ATP from high-energy phosphate bonds, e.g., acetate kinase, creatine kinase, etc. Such enzymes may be present in the extracts used for translation, or may be added to the reaction mix. Such synthetic reaction systems are well-known in the art, and have been described in the literature. The system generally includes a nucleic acid template that encodes a protein of interest. The nucleic acid template is an RNA molecule (e.g., mRNA) or a nucleic acid that encodes an mRNA (e.g., RNA, DNA) and be in any form (e.g., linear, circular, supercoiled, single stranded, double stranded, etc.). Nucleic acid templates guide production of the desired protein.

Turning to the analyte-of-interest, the compositions and methods described here can be used to detect any analyte-of-interest, including nucleic acid molecule-based analytes capable of binding and disrupting the RNA toehold switch molecules described herein, as well as small molecule, peptide, or polypeptide analytes that can bind an allosteric transcription factor (engineered or naturally occurring). In some embodiments, the analyte comprises at least one of a DNA molecule, an RNA molecule, a small molecule, a lipid, a peptide, a polypeptide, a protein, a glycoprotein, and the like. In some embodiments, the analyte is from a pathogenic organism selected from the group consisting of bacteria, viruses, protozoa, worms, fungi, and the like. In a particular embodiment, the analyte comprises RNA from a virus. In some embodiments, the virus is a SARS-CoV-2.

In some embodiments, the RNA molecule capable of binding an analyte-of-interest comprises the nucleic acid sequence: GGGNNNNNNNNNNNNNNNNNNNNNNNNNNNNNNAACAGAGGAGANNNNNNAU GNNNNNNNNNAACGGUAGCGCAGGUAGCGGCAUAUG (SEQ ID NO: 1). In some embodiments, the RNA molecule comprises a nucleic acid sequence that is at least 70% identical to SEQ ID NO: 1. In some embodiments, the RNA molecule comprises a nucleic acid sequence that is at least 75% identical to SEQ ID NO: 1. In some embodiments, the RNA molecule comprises a nucleic acid sequence that is at least 80% identical to SEQ ID NO: 1. In some embodiments, the RNA molecule comprises a nucleic acid sequence that is at least 85% identical to SEQ ID NO: 1. In some embodiments, the RNA molecule comprises a nucleic acid sequence that is at least 90% identical to SEQ ID NO: 1. In some embodiments, the RNA molecule comprises a nucleic acid sequence that is at least 95% identical to SEQ ID NO: 1.

In other embodiments, the RNA molecule comprises a nucleic acid sequence selected from the group consisting of SEQ ID NOs: 2-19. In some embodiments, the RNA molecule comprises a nucleic acid sequence that is at least 70% identical to any of SEQ ID NOs: 2-19. In some embodiments, the RNA molecule comprises a nucleic acid sequence that is at least 75% identical to any of SEQ ID NOs: 2-19. In some embodiments, the RNA molecule comprises a nucleic acid sequence that is at least 80% identical to any of SEQ ID NOs: 2-19. In some embodiments, the RNA molecule comprises a nucleic acid sequence that is at least 85% identical to any of SEQ ID NOs: 2-19. In some embodiments, the RNA molecule comprises a nucleic acid sequence that is at least 90% identical to any of SEQ ID NOs: 2-19. In some embodiments, the RNA molecule comprises a nucleic acid sequence that is at least 95% identical to any of SEQ ID NOs: 2-19.

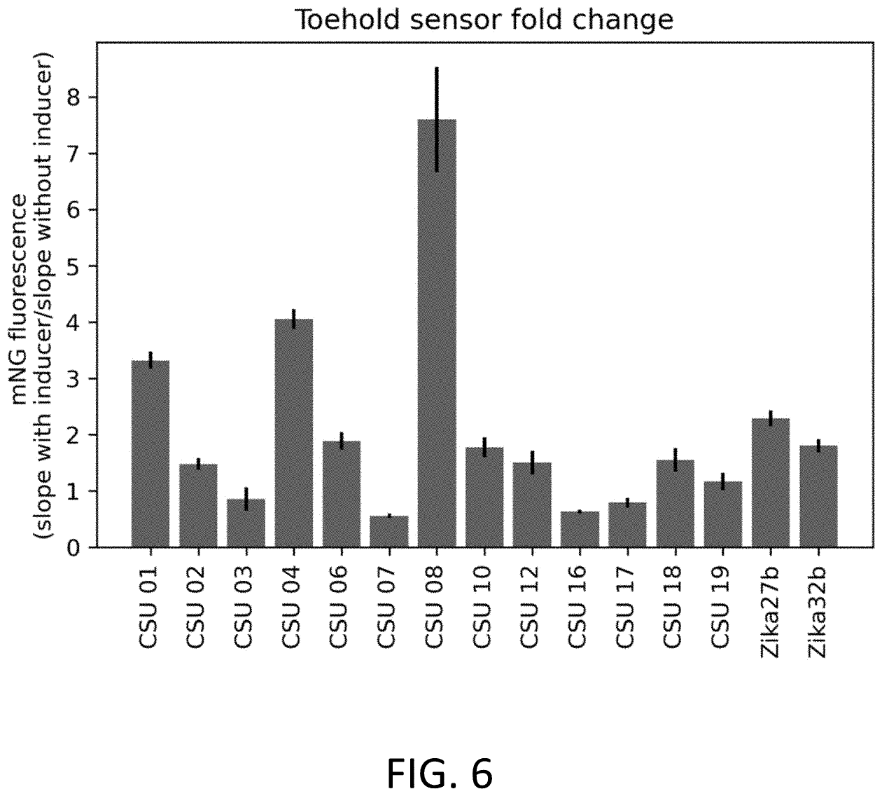

Embodiments of the present disclosure also include a method for detecting an analyte that includes combining a composition comprising any of the detection components and any of the signal amplification components described herein with a biological sample, and measuring or detecting a signal produced by the signal amplification component (FIGS. 6-8). In some embodiments, the presence of the analyte in the biological sample causes expression of an initiator protein and subsequent activation of the signal amplification component.

As would be recognized by one of ordinary skill in the art based on the present disclosure, the compositions and methods described herein can be used with any sample. However, in some embodiments, the biological sample comprises a blood sample, a plasma sample, a serum sample, a cerebral spinal fluid sample, a saliva sample, a tear sample, a urine sample, a fecal sample, a cell sample, a tissue sample, a water sample, and/or a plant sample.

In some embodiments, the method further comprises quantifying the signal and determining a concentration of the analyte in the biological sample. In some embodiments, the analyte comprises viral RNA from SARS-CoV-2. In some embodiments, the signal is a fluorescent signal, a bioluminescent signal, a chemical signal, an electrochemical signal, or a colorimetric signal. In some embodiments, the analyte is present in the sample at a concentration ranging from about 1 fM to about 1 μM. In some embodiments, the analyte is present in the sample at a concentration ranging from about 500 fM to about 1 μM. In some embodiments, the analyte is present in the sample at a concentration ranging from about 1 pM to about 1 μM. In some embodiments, the analyte is present in the sample at a concentration ranging from about 500 pM to about 1 μM. In some embodiments, the analyte is present in the sample at a concentration ranging from about 1 nM to about 1 μM. In some embodiments, the analyte is present in the sample at a concentration ranging from about 500 nM to about 1 μM. In some embodiments, the analyte is present in the sample at a concentration ranging from about 1 fM to about 500 nM. In some embodiments, the analyte is present in the sample at a concentration ranging from about 1 fM to about 1 nM. In some embodiments, the analyte is present in the sample at a concentration ranging from about 1 fM to about 500 pM. In some embodiments, the analyte is present in the sample at a concentration ranging from about 1 fM to about 1 pM. In some embodiments, the analyte is present in the sample at a concentration ranging from about 1 pM to about 1 nM.

Embodiments of the present disclosure also include a kit comprising any of the compositions described herein and instructions for performing an analyte detection assay. In some embodiments, the kit comprises one or more components of a cell free protein expression system and/or a reaction buffer.

As described above, embodiments of the present disclosure also include a composition for performing an analyte detection assay in which the detection component includes an allosteric transcription factor. In accordance with these embodiments, the composition includes a detection component comprising an allosteric transcription factor comprising an analyte recognition domain, and a DNA molecule comprising an allosteric transcription factor binding domain, a ribosome binding site, and an initiator protein domain; and a signal amplification component responsive to expression of an initiator protein encoded by the initiator protein domain. In some embodiments, the presence of the analyte causes expression of the initiator protein and subsequent activation of the signal amplification component.

In some embodiments, the allosteric transcription factor is selected from the group consisting of: (i) a TetR transcription factor selected from TetR, MphR, QacR, and TtgR, and any variants or derivates thereof; (ii) a multiple antibiotic resistance repressor (MarR) transcription factors selected from OtrR, CtcS, MobR, and HucR, and any variants or derivates thereof; (iii) an ArsR/SmtB transcriptional regulator selected from SmtB and CadC, and any variants or derivates thereof; (iv) a CsoR/RcnR transcriptional regulator selected from CsoR, and any variants or derivates thereof; (v) a MerR transcriptional regulator, and any variants or derivates thereof; (vi) a Fur transcriptional regulator, and any variants or derivates thereof; (vii) a DtxR transcriptional regulator, and any variants or derivates thereof; (viii) a NikR transcriptional regulator, and any variants or derivates thereof; and (ix) a xylR transcriptional activator acting on toluene, xylene, benzene, and any variants or derivatives thereof. As would be recognized by one of ordinary skill in the art based on the present disclosure, any allosteric transcription factor can be used with the compositions and methods of the present disclosure.

### 3. MATERIALS AND METHODS

Design of Toehold Switch Sensors. Toehold switches were designed from the SARS-CoV-2 Wuhan strain (NCBI Reference Sequence NC_045512.2) with the following parameters (FIG. 3): a 3 nucleotide (nt) GGG T7 promoter enhancer sequence followed by a 12 nt toehold with an 18 nt stem sequence complementary to a 30 nt RNA inducer sequence. This was followed by an 11 nt loop containing the ribosome binding site (RBS) then 18 nt complementary to the first 18 nt stem sequence except for a start codon which occurs 6 nt after the loop. Toehold Switches were designed using an iterative process either manually or with the STORM toehold switch generator software (storm-toehold.herokuapp.com). An 8 amino acid linker sequence NGSAGSGH was included in all designs between the toehold switch and the reporter gene. A total of 19 toehold switches were designed for testing (Table 1).

mNeonGreen Expression Plasmid Design and Toehold Switch Sensor Synthesis.

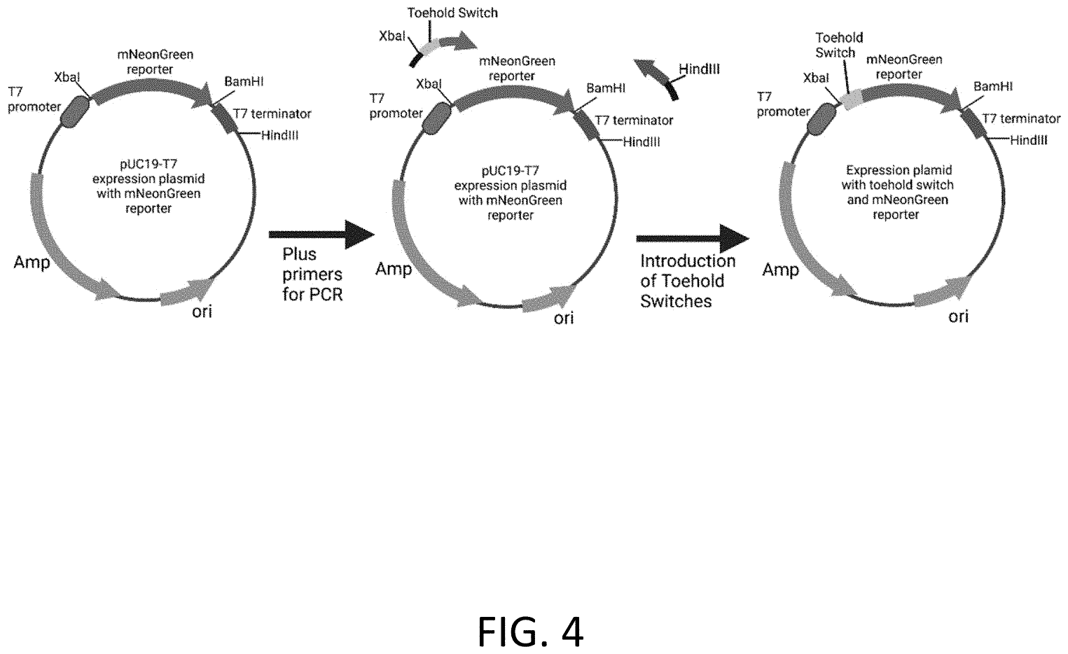

A DNA fragment containing a T7 phage promoter, an E. coli codon-optimized mNeonGreen gene, and a T7 terminator was synthesized by TWIST Biosciences (San Francisco, Calif.) in pTwist Chlor MC. An XbaI and a BamHI site were included after the promoter and before the terminator, respectively, for directional cloning. A HindIII site 3′ to the terminator was added for cloning and/or plasmid linearizing. The construct was cloned into pUC19 to generate a high copy pUC19-mNeonGreen expression plasmid shown below (FIG. 4).

Toehold switches were incorporated into the mNeonGreen expression plasmid for testing with RNA inducers. Toehold switches were synthesized as part of a 117 nt forward primer using an ultramer DNA oligonucleotide (IDT, Coralville, Iowa) containing an XbaI site and a 19 nt region at the 3′ end complementary to the mNeonGreen gene. A reverse primer complementary to the 3′ end of the T7 terminator and incorporating a HindIII site was used with the ultramer oligonucleotide for PCR. Either Herculase II (Agilent, Santa Clara, Calif.) or Phusion (NEB, Ipswich, Mass.) high fidelity polymerases were used for the PCR reactions, according to the manufacturer's protocols.

RNA inducers were based on the inducer design of Green et al., and included a 30 nt inducer sequence flanked by stem loop structure at the 5′ and 3′ ends of the inducer. Inducers were constructed as follows: 1) a GGG T7 enhancer, 2) a 5′ stem-loop consisting of a 10 nt stem with a 6 nt loop and a second 10 nt complementary stem (26 nt total), 3) a 3 nt spacer followed by the 30 nt inducer RNA sequence complementary to its respective toehold switch, 4) a 3 nt spacer, and 5) the T7 Terminator which adopts a 14 nt stem and an 8 nt loop structure. The size of the RNA inducer with flanking stem-loops GGG enhancer, and XbaI cloning site was 115 nt.

Inducer sequences were constructed in pUC19 using a process similar to that used for cloning the toehold switches (FIG. 4). The inducer sequences were synthesized into an ultramer oligonucleotide (IDT, Coralville, Iowa) with an XbaI site and 17 nucleotides at the 3′ end complementary to the 5′ end of the T7 terminator. A reverse primer was constructed that was complementary to the region downstream of the T7 terminator and included the HindIII site. XbaI and HindIII restriction sites were incorporated into the primers for cloning the PCR product into the pUC19-T7 expression plasmid.

TEV Protease Cloning. To generate the TEV protease amplification system, a construct encoding the T7 promoter, the CSU 08 toehold switch, an E. coli codon-optimized version of the Tobacco Etch Virus (TEV) protease gene and T7 terminator was synthesized in pUC57-Amp by SynBio Technologies (Monmouth Junction, N.J.).

Alternative toehold switches were fused to TEV protease by using an ultramer oligonucleotide with the TEV protease gene incorporated. High fidelity PCR (Herculase II fusion DNA polymerase, Agilent) was then used to join and amplify the toehold switch and the TEVprotease. The PCR product was cloned back into the pUC57 T7 expression plasmid using XbaI and BamHI restriction sites flanking the 5′ UTR and 3′ end of the TEV protease gene.

Screening of Toehold Switches. To identify the toehold switch that gave the highest readout signal, the 115 nt RNA Inducer-expression plasmid was added to the cell free reaction (CFPS,) and co-expressed with an equal concentration of the toehold switch expression plasmid. Cell-free protein synthesis (CFPS) reactions driven by a T7 RNA Polymerase expression system were assembled according to the manufacturer's instructions using the NEBExpress Cell-free E. coli Protein Synthesis System #E5360 (NEB, Ipswich, Mass.). This kit contains the T7 RNA polymerase expression system, including the necessary energy source, amino acids, translation factors, ribosomes, and a murine RNase inhibitor.

For toehold switch screening, CFPS reagents were mixed with plasmids containing the toehold switch and inducer (each at 5 nM final concentration). Reactions were conducted in 384-well plates in triplicate in 25 μl total volume per well. All mNeonGreen fluorescence detection reactions were run at 37° C. to maximize the E. coli T7 expression system. Two Zika toehold switches as reported by Pardee et al., and coworkers served as positive controls in preliminary screens.

Imaging. A BioTek Synergy H-1 microplate reader was used for imaging all cell-free reactions in Greiner non-binding, black-bottom, black-sided 384 well plates (Grenier Bio-One, Monroe, N.C.). Samples were tape-sealed with Bio-Rad Microseal PCR Plate Sealing Film Adhesive #MSB1001 (Bio-Rad, Hercules, Calif.) prior to imaging. A time-course plot of fluorescence activity data for each cell-free reaction was recorded in 5-minute increments over a period of several hours. An excitation:emission spectrum of 485 nm:528 nm was used for mNeonGreen detection and 490 nm:520 nm for fluorescein detection.

All time-course plots included linear shaking before every fluorescence reading to ensure well-mixed reactions. The reactions were allowed to run for several hours to determine the time of the most linear increase in fluorescence.

Calculation of Fold Change. To calculate fold change, the methodology of Pardee et al. was followed, as described herein. The most linear section of the fluorescence detection curve was typically between 60 minutes and 120 minutes of the time course plot. Therefore, the average slope for each technical replicate was calculated between those two time points. A sample average and sample standard deviation was calculated by averaging the three technical replicate slopes for each sample, including that for the uninduced control sample (lacking the inducer-containing plasmid). Each of the experimental average slopes was then divided by the uninduced control average slope. The resulting ratio shows the relative increase in fluorescence signal by each experimental sample compared to the uninduced control and is referred to as the ‘fold-change.’ For plotting purposes, the time course reaction raw data was adjusted by subtracting the average background fluorescence calculated from the no-plasmid negative control sample, and a three-point moving average was used to smooth the curve.

RNA Synthesis of the 115 Nucleotide and 5,100 Nucleotide SARS-CoV-Inducers for LOD Experiments. Plasmid-free SARS-CoV2 RNA inducers were used in limit of detection experiments. They were produced from either a HindIII-linearized pUC19 115 nt inducer expression plasmid or from a subgenomic SARS-CoV2 PCR amplification product flanked by a T7 promoter and Terminator

For synthesis of the 115 nt inducer RNA, HindIII-linearized RNA inducer expression plasmid (1 μg) was added to a 20 μl reaction volume using the HiScribe T7 High Yield RNA Synthesis Kit (NEB, Ipswich, Mass. #E2040S). The 115 nt inducer RNA was transcribed from the T7 promoter of the inducer-plasmid using the manufacturer's protocol, with the addition of 1 μl of murine RNase inhibitor (NEB, Ipswich, Mass. #M0314S). After incubation at 37° C. for 2 hr, plasmid DNA was removed from the reaction using the TURBO DNase kit (Thermo Fisher Scientific, Waltham, Mass. #AM1907) according to the manufacturer's protocol.

To produce the 5,100 nt RNA inducer, a pUC57 plasmid containing a 6.8 kb cDNA fragment corresponding to nucleotide position 1 to 6844 of the SARS-CoV-2 genome (gift from Brian Geiss, Colorado State University) was used as the template. This region was chosen since it encompassed both regions 1 (nt 1918-1933) and 2 (nt 1865-2018) used for inducer switches and contained a larger region of the SARS-CoV-2 genome.

Ultramer oligonucleotides containing a T7 promoter in the forward primer and a T7 terminator in the reverse primer and the pUC57-6.8 kb template were used to amplify a 5,100 nt PCR product. The amplified PCR product (1 μg per 20 μl reaction volume) was mixed with reaction buffer (1×), dNTPs (final concentration 12 mM), PEG 8000 (final concentration 2%), 1 μl of Murine RNAse Inhibitor and 2 μl of T7 RNA polymerase and incubated at 37° C. for 30 minutes. The template DNA was removed from the RNA reaction using the TURBO DNase kit (InVitrogen, Carlsbad, Calif.) according to manufacturer's protocol. The quality and size of the RNA was confirmed on a 2% w/v TAE agarose gel containing 1% v/v Clorox bleach stained with 0.5 mg/ml ethidium bromide according to Aranda et al.

Limit of Detection Assays. Limit of detection (LOD) assays were performed using the CSU 08 toehold switch and its complementary RNA inducer in both the mNeonGreen expression plasmid and in the TEV protease amplification system. Triplicate 25 μl liquid reactions in a 364-well plate were used to test each sensor, with each toehold switch plasmid at a final concentration of 5 nM. A ten-fold dilution series of complementary inducer RNA (115 nt and 5,100 nt) was performed in triplicate using NEB PURExpress In Vitro Protein Synthesis Kit #E6800L (NEB, Ipswich, Mass.) and a constant concentration of plasmid. All mNeonGreen LOD reactions were conducted at 37° C. to optimize the T7 expression system. All TEV protease reactions were conducted at a temperature of 30° C., due to the optimum range of TEV assay activity.

The TEV reporter enzymatic reaction was visualized by the cleavage of a TEV-specific fluorescein-quencher recombinant protein substrate provided by the Anaspec SensoLyte 520 TEV Protease Assay Kit #AS-72227 (AnaSpec, Fremont, Calif.). The manufacturer's instructions for this kit were modified to incorporate a 10% v/v buffer-substrate solution into each 25 μl TEV reporter toehold switch reaction. This resulted in a 1× final concentration of the fluorescein-quencher substrate for each enzymatic toehold reaction.

Sequences. The present disclosure provides the following nucleic acid and amino acid sequences that are relevant to one or more embodiments of the methods and compositions described herein.

Consensus RNA sequence for toehold switches described herein:

(SEQ ID NO: 1; see FIG. 3)
GGGNNNNNNNNNNNNNNNNNNNNNNNNNNNNNNAACAGAGGAGANNN
NNNAUGNNNNNNNNNAACGGUAGCGCAGGUAGCGGCAUAUG.

TABLE 1
Analysis of the predicted binding (on/off state) of the inducer
RNA to the toehold switch and the fold-change
in mNeonGreen fluorescence in screening experiments.

| | Genomic | Predicted | Fold Change | Inducer | Toehold |
|---|---|---|---|---|---|
| Toehold | Region | ON/OFF | of CFPS | RNA | Switch |
| Name | targeted | (transfer) | Fluorescence | sequence | sequence |
| CSU 01 | 1988-2017 | 0.401 | 3.5 | CAGTATTCACTG | CATAGCATCAATGAGTC |
| | (region 2: | | | AGACTCATTGAT | TCAGTGAATACTGAAC |
| | 1865-2018) | | | GCTATG | AGAGGAGACAGTATAT |
| | | | | (SEQ ID NO: 22) | GCTGAGACTC |
| | | | | | (SEQ ID NO: 2) |
| CSU 02 | 1931-1960 | 0.358 | 1.5 | CAAAATTCTGTG | GGCCTTCTGTAAAACAC |
| | (region 2) | | | CGTGTTTTACAG | GCACAGAATTTTGAAC |
| | | | | AAGGCC | AGAGGAGACAAAATAT |
| | | | | (SEQ ID NO: 23) | GGTGCGTGTT |
| | | | | | (SEQ ID NO: 3) |
| CSU 03 | 1955-1984 | 0.358 | 0.9 | AAGGCCGCTATA | AATTCCATCTAGTATTG |
| | (region 2) | | | ACAATACTAGAT | TTATAGCGGCCTTAACA |
| | | | | GGAATT | GAGGAGAAAGGCCATG |
| | | | | (SEQ ID NO: 24) | ATAACAATA |
| | | | | | (SEQ ID NO: 4) |
| CSU 04 | 1964-1993 | 0.353 | 4.1 | ATAACAATACTA | ATACTGTGAAATTCCAT |
| | (region 2) | | | GATGGAATTTCA | CTAGTATTGTTATAACA |
| | | | | CAGTAT | GAGGAGAATAACAATG |
| | | | | (SEQ ID NO: 25) | CTAGATGGA |
| | | | | | (SEQ ID NO: 5) |
| CSU 05 | 1944-1973 | 0.351 | 2.2 | GTGTTTTACAGA | GTATTGTTATAGCGGCC |
| | (region 2) | | | AGGCCGCTATAA | TTCTGTAAAACACAACA |
| | | | | CAATAC | GAGGAGAGTGTTTATG |
| | | | | (SEQ ID NO: 26) | AGAAGGCCG |
| | | | | | (SEQ ID NO: 6) |
| CSU 06 | 1865-1894 | 0.349 | 1.9 | TATGCATTTGCA | AACACGAGCAGCCTCT |
| | (region 2) | | | TCAGAGGCTGCT | GATGCAAATGCATAAA |
| | | | | CGTGTT | CAGAGGAGATATGCAA |
| | | | | (SEQ ID NO: 27) | TGGCATCAGAG |
| | | | | | (SEQ ID NO: 7) |
| CSU 07 | 1932-1961 | 0.345 | 0.6 | AAAATTCTGTGC | CGGCCTTCTGTAAAACA |
| | (region 2) | | | GTGTTTTACAGA | CGCACAGAATTTTAACA |
| | | | | AGGCCG | GAGGAGAAAAATTATG |
| | | | | (SEQ ID NO: 28) | TGCGTGTTT |
| | | | | | (SEQ ID NO: 8) |
| CSU 08 | 1900-1929 | 0.343 | 7.5 | ATCAATTTTCTC | GCAGTTTCAAGAGTGC |
| | (region 1: | | | CCGCACTCTTGA | GGGAGAAAATTGATAA |
| | 1918- | | | AACTGC | CAGAGGAGAATCAATA |
| | 1933) | | | (SEQ ID NO: 29) | TGCTCCCGCAC |
| | | | | | (SEQ ID NO: 9) |
| CSU 09 | 1901-1930 | 0.284 | 4.2 | TCAATTTTCTCC | AGCAGTTTCAAGAGTG |
| | (region 1) | | | CGCACTCTTGAA | CGGGAGAAAATTGAAA |
| | | | | ACTGCT | CAGAGGAGATCAATTA |
| | | | | (SEQ ID NO: 30) | TGTCCCGCACT |
| | | | | | (SEQ ID NO: 10) |
| CSU 10 | 1919-1948 | 0.225 | 1.8 | CTTGAAACTGCT | AACACGCACAGAATTTT |
| | (region 1) | | | CAAAATTCTGTG | GAGCAGTTTCAAGAAC |
| | | | | CGTGTT | AGAGGAGACTTGAAAT |
| | | | | (SEQ ID NO: 31) | GGCTCAAAAT |
| | | | | | (SEQ ID NO: 11) |
| CSU 11 | 1904-1933 | 0.124 | 0.95 | ATTTTCTCCCGC | TTGAGCAGTTTCAAGAG |
| | (region 1) | | | ACTCTTGAAACT | TGCGGGAGAAAATAAC |
| | | | | GCTCAA | AGAGGAGAATTTTCAT |
| | | | | (SEQ ID NO: 32) | GCGCACTCTT |
| | | | | | (SEQ ID NO: 12) |
| CSU 12 | 1907-1936 | 0.059 | 1.5 | TTCTCCCGCACT | ATTTTGAGCAGTTTCAA |
| | (region 1) | | | CTTGAAACTGCT | GAGTGCGGGAGAAAAC |
| | | | | CAAAAT | AGAGGAGATTCTCCAT |
| | | | | (SEQ ID NO: 33) | GACTCTTGAA |
| | | | | | (SEQ ID NO: 13) |
| CSU 13 | 1904-1937 | 0.119 | 2.2 | ATTTTCTCCCGC | AATTTTGAGCAGTTTCA |
| | (region 1) | | | ACTCTTGAAACT | AGAGTGCGGGAGAAAA |
| | | | | GCTCAAAATT | TAACAGAGGAGAATTT |
| | | | | (SEQ ID NO: 34) | TCATGCGCAC |
| | | | | | (SEQ ID NO: 14) |
| CSU 15 | 1915-1944 | 0.043 | 0.95 | CACTCTTGAAAC | CGCACAGAATTTTGAGC |
| | (region 1) | | | TGCTCAAAATTC | AGTTTCAAGAGTGAAC |
| | | | | TGTGCG | AGAGGAGACACTCTAT |
| | | | | (SEQ ID NO: 35) | GAACTGCTCA |
| | | | | | (SEQ ID NO: 15) |
| CSU 16 | 1918-1947 | 0.068 | 0.6 | TCTTGAAACTGC | ACACGCACAGAATTTTG |
| | (region 1) | | | TCAAAATTCTGT | AGCAGTTTCAAGAAAC |
| | | | | GCGTGT | AGAGGAGATCTTGAAT |
| | | | | (SEQ ID NO: 36) | GTGCTCAAAA |
| | | | | | (SEQ ID NO: 16) |
| CSU 17 | 1881-1910 | 0.057 | 0.8 | AGGCTGCTCGTG | AGAAAATTGATCGTAC |
| | (region 2) | | | TTGTACGATCAA | AACACGAGCAGCCTAA |
| | | | | TTTTCT | CAGAGGAGAAGGCTGA |
| | | | | (SEQ ID NO: 37) | TGGTGTTGTAC |
| | | | | | (SEQ ID NO: 17) |
| CSU 18 | | | | | |
| | (region 2) | | | GTACGATCAATT | ACAACACGAGCAGCAA |
| | | | | TTCTCC | CAGAGGAGAGCTGCTA |
| | | | | (SEQ ID NO: 38) | TGGTTGTACGA |
| | | | | | (SEQ ID NO: 18) |
| CSU 19 | 1941-1970 | 0.17 | 1.1 | TGCGTGTTTTAC | TTGTTATAGCGGCCTTC |
| | (region2) | | | AGAAGGCCGCT | TGTAAAACACGCAAAC |
| | | | | ATAACAA | AGAGGAGATGCGTGAT |
| | | | | (SEQ ID NO: 39) | GTACAGAAGG |
| | | | | | (SEQ ID NO: 19) |
| 27B Zika | 1373-1408 | 0.162 | 2.1 | GACACAGGACA | TTTCGCTCTATTCTCAT |
| Control | | | | TGAAACTGATGA | CAGTTTCATGTCCTGTG |
| | | | | GAATAGAGCGA | TCGGACTTTAGAACAG |
| | | | | AA | AGGAGATAAAGATGGA |
| | | | | (SEQ ID NO: 40) | CACAGGACA |
| | | | | | (SEQ ID NO: 20) |
| 32B Zika | 7188-7222 | 0.213 | 1.9 | TTTTGCTCGTGG | TGGGATCAAGTACATGT |
| Control | | | | CGCACTACATGT | AGTGCGCCACGAGCAA |
| | | | | ACTTGATCCCA | AAGGACTTTAGAACAG |
| | | | | (SEQ ID NO: 41) | AGGAGATAAAGATGTT |
| | | | | | TTGCTCGTG |
| | | | | | (SEQ ID NO: 21) |

Amino acid sequence of a TEV-specific protease cleavage site ENLYFQG (SEQ ID NO: 42).
pUC19 T7 promoter mNeonGreen T7 terminator (T7 promoter, XbaI, mNeonGreen,
BamHI,T7 terminator, HindIII):
(SEQ ID NO: 43)
agttcggtgtaggtcgttcgctccaagctgggctgtgtgcacgaaccccccgttcagcccgaccgctgcgccttatccggtaactatcg
tcttgagtccaacccggtaagacacgacttatcgccactggcagcagccactggtaacaggattagcagagcgaggtatgtaggcgg
tgctacagagttcttgaagtggtggcctaactacggctacactagaagaacagtatttggtatctgcgctctgctgaagccagttaccttc
ggaaaaagagttggtagctcttgatccggcaaacaaaccaccgctggtagcggtggtttttttgtttgcaagcagcagattacgcgcag
aaaaaaaggatctcaagaagatcctttgatcttttctacggggtctgacgctcagtggaacgaaaactcacgttaagggattttggtcatg
agattatcaaaaaggatcttcacctagatccttttaaattaaaaatgaagttttaaatcaatctaaagtatatatgagtaaacttggtctgaca
gttaccaatgcttaatcagtgaggcacctatctcagcgatctgtctatttcgttcatccatagttgcctgactccccgtcgtgtagataacta
cgatacgggagggcttaccatctggccccagtgctgcaatgataccgcgagacccacgctcaccggctccagatttatcagcaataaa
ccagccagccggaagggccgagcgcagaagtggtcctgcaactttatccgcctccatccagtctattaattgttgccgggaagctaga
gtaagtagttcgccagttaatagtttgcgcaacgttgttgccattgctacaggcatcgtggtgtcacgctcgtcgtttggtatggcttcattc
agctccggttcccaacgatcaaggcgagttacatgatcccccatgttgtgcaaaaaagcggttagctccttcggtcctccgatcgttgtc
agaagtaagttggccgcagtgttatcactcatggttatggcagcactgcataattctcttactgtcatgccatccgtaagatgcttttctgtg
actggtgagtactcaaccaagtcattctgagaatagtgtatgcggcgaccgagttgctcttgcccggcgtcaatacgggataataccgc
gccacatagcagaactttaaaagtgctcatcattggaaaacgttcttcggggcgaaaactctcaaggatcttaccgctgttgagatccag
ttcgatgtaacccactcgtgcacccaactgatcttcagcatcttttactttcaccagcgtttctgggtgagcaaaaacaggaaggcaaaat
gccgcaaaaaagggaataagggcgacacggaaatgttgaatactcatactcttcctttttcaatattattgaagcatttatcagggttattgt
ctcatgagcggatacatatttgaatgtatttagaaaaataaacaaataggggttccgcgcacatttccccgaaaagtgccacctgacgtct
aagaaaccattattatcatgacattaacctataaaaataggcgtatcacgaggccctttcgtctcgcgcgtttcggtgatgacggtgaaaa
cctctgacacatgcagctcccggagacggtcacagcttgtctgtaagcggatgccgggagcagacaagcccgtcagggcgcgtcag
cgggtgttggcgggtgtcggggctggcttaactatgcggcatcagagcagattgtactgagagtgcaccatatgcggtgtgaaatacc
gcacagatgcgtaaggagaaaataccgcatcaggcgccattcgccattcaggctgcgcaactgttgggaagggcgatcggtgcggg
cctcttcgctattacgccagctggcgaaagggggatgtgctgcaaggcgattaagttgggtaacgccagggttttcccagtcacgacgt
tgtaaaacgacggccagtgaattcTAATACGACTCACTATAGGGTCTAGAAATAATTTTGTTTA
ACTTTAAGAAGGAGATATACATATGGTGAGCAAGGGCGAGGAGGATAACATGGC
CTCTCTCCCAGCGACACATGAGTTACACATCTTTGGCTCCATCAACGGTGTGGAC
TTTGACATGGTGGGTCAGGGCACCGGCAATCCAAATGATGGTTATGAGGAGTTA
AACCTGAAGTCCACCAAGGGTGACCTCCAGTTCTCCCCCTGGATTCTGGTCCCTC
ATATCGGGTATGGCTTCCATCAGTACCTGCCCTACCCTGACGGGATGTCGCCTTT
CCAGGCCGCCATGGTAGATGGCTCCGGATACCAAGTCCATCGCACAATGCAGTTT
GAAGATGGTGCCTCCCTTACTGTTAACTACCGCTACACCTACGAGGGAAGCCACA
TCAAAGGAGAGGCCCAGGTGAAGGGGACTGGTTTCCCTGCTGACGGTCCTGTGA
TGACCAACTCGCTGACCGCTGCGGACTGGTGCAGGTCGAAGAAGACTTACCCCA
ACGACAAAACCATCATCAGTACCTTTAAGTGGAGTTACACCACTGGAAATGGCA
AGCGCTACCGGAGCACTGCGCGGACCACCTACACCTTTGCCAAGCCAATGGCGG
CTAACTATCTGAAGAACCAGCCGATGTACGTGTTCCGTAAGACGGAGCTCAAGC
ACTCCAAGACCGAGCTCAACTTCAAGGAGTGGCAAAAGGCCTTTACCGATGTGA
TGGGCATGGACGAGCTGTACAAGTAATGAGGATCCCGGGAATTCTCGAGTAAGG
TTAACCTGCAGGAGGCCTTTAATTAAGGTGGTGCGGCCGCGCTAGCGGTCCCGGG
GGATCGATCCGGCTGCTAACAAAGCCCGAAAGGAAGCTGAGTTGGCTGCTGCCA
CCGCTGAGCAATAACTAGCATAACCCCTTGGGGCCTCTAAACGGGTCTTGAGGG
GTTTTTTGCTGAAAGGAGGAACTATATCCGGaagcttggcgtaatcatggtcatagctgtttcctgtgtgaa
attgttatccgctcacaattccacacaacatacgagccggaagcataaagtgtaaagcctggggtgcctaatgagtgagctaactcaca
ttaattgcgttgcgctcactgcccgctttccagtcgggaaacctgtcgtgccagctgcattaatgaatcggccaacgcgcggggagag
gcggtttgcgtattgggcgctcttccgcttcctcgctcactgactcgctgcgctcggtcgttcggctgcggcgagcggtatcagctcact
caaaggcggtaatacggttatccacagaatcaggggataacgcaggaaagaacatgtgagcaaaaggccagcaaaaggccagga
accgtaaaaaggccgcgttgctggcgtttttccataggctccgcccccctgacgagcatcacaaaaatcgacgctcaagtcagaggtg
gcgaaacccgacaggactataaagataccaggcgtttccccctggaagctccctcgtgcgctctcctgttccgaccctgccgcttacc
ggatacctgtccgcctttctcccttcgggaagcgtggcgctttctcatagctcacgctgtaggtatctc.
pUC19 T7 promoter 115 nt CSU 08 inducer RNA with T7 terminator (T7 promoter,
XbaI 5’ stem loop, 30 nucleotide trigger, T7 terminator, HindIII):
(SEQ ID NO: 44)
agttcggtgtaggtcgttcgctccaagctgggctgtgtgcacgaaccccccgttcagcccgaccgctgcgccttatccggtaactatcg
tctt,gagtccaacccggtaagacacgacttatcgccactggcagcagccactggtaacaggattagcagagcgaggtatgtaggcg
gtgctacagagttcttgaagtggtggcctaactacggctacactagaagaacagtatttggtatctgcgctctgctgaagccagttacctt
cggaaaaagagttggtagctcttgatccggcaaacaaaccaccgctggtagcggtggtttttttgtttgcaagcagcagattacgcgca
gaaaaaaaggatctcaagaagatcctttgatcttttctacggggtctgacgctcagtggaacgaaaactcacgttaagggattttggtcat
gagattatcaaaaaggatcttcacctagatccttttaaattaaaaatgaagttttaaatcaatctaaagtatatatgagtaaacttggtctgac
agttaccaatgcttaatcagtgaggcacctatctcagcgatctgtctatttcgttcatccatagttgcctgactccccgtcgtgtagataact
acgatacgggagggcttaccatctggccccagtgctgcaatgataccgcgagacccacgctcaccggctccagatttatcagcaataa
accagccagccggaagggccgagcgcagaagtggtcctgcaactttatccgcctccatccagtctattaattgttgccgggaagctag
agtaagtagttcgccagttaatagtttgcgcaacgttgttgccattgctacaggcatcgtggtgtcacgctcgtcgtttggtatggcttcatt
cagctccggttcccaacgatcaaggcgagttacatgatcccccatgttgtgcaaaaaagcggttagctccttcggtcctccgatcgttgt
cagaagtaagttggccgcagtgttatcactcatggttatggcagcactgcataattctcttactgtcatgccatccgtaagatgcttttctgt
gactggtgagtactcaaccaagtcattctgagaatagtgtatgcggcgaccgagttgctcttgcccggcgtcaatacgggataataccg
cgccacatagcagaactttaaaagtgctcatcattggaaaacgttcttcggggcgaaaactctcaaggatcttaccgctgttgagatcca
gttcgatgtaacccactcgtgcacccaactgatcttcagcatcttttactttcaccagcgtttctgggtgagcaaaaacaggaaggcaaaa
tgccgcaaaaaagggaataagggcgacacggaaatgttgaatactcatactcttcctttttcaatattattgaagcatttatcagggttattg
tctcatgagcggatacatatttgaatgtatttagaaaaataaacaaataggggttccgcgcacatttccccgaaaagtgccacctgacgtc
taagaaaccattattatcatgacattaacctataaaaataggcgtatcacgaggccctttcgtctcgcgcgtttcggtgatgacggtgaaa
acctctgacacatgcagctcccggagacggtcacagcttgtctgtaagcggatgccgggagcagacaagcccgtcagggcgcgtca
gcgggtgttggcgggtgtcggggctggcttaactatgcggcatcagagcagattgtactgagagtgcaccatatgcggtgtgaaatac
cgcacagatgcgtaaggagaaaataccgcatcaggcgccattcgccattcaggctgcgcaactgttgggaagggcgatcggtgcgg
gcctcttcgctattacgccagctggcgaaagggggatgtgctgcaaggcgattaagttgggtaacgccagggttttcccagtcacgac
gttgtaaaacgacggccagtgaattcTAATACGACTCACTATAGGGTCTAGAGGGACATACGGA
CTCACGTGTCCGTATGTCAAatcaattttctcccgcactcttgaaactgcACTAACCCCTTGGGGCCT
CTAAACGGGTCTTGAGGGGTTTTTTGctgaaaggaggaactatatccggaagcttggcgtaatcatggtcata
gctgtttcctgtgtgaaattgttatccgctcacaattccacacaacatacgagccggaagcataaagtgtaaagcctggggtgcctaatg
agtgagctaactcacattaattgcgttgcgctcactgcccgctttccagtcgggaaacctgtcgtgccagctgcattaatgaatcggcca
acgcgcggggagaggcggtttgcgtattgggcgctcttccgcttcctcgctcactgactcgctgcgctcggtcgttcggctgcggcga
gcggtatcagctcactcaaaggcggtaatacggttatccacagaatcaggggataacgcaggaaagaacatgtgagcaaaaggcca
gcaaaaggccaggaaccgtaaaaaggccgcgttgctggcgtttttccataggctccgcccccctgacgagcatcacaaaaatcgacg
ctcaagtcagaggtggcgaaacccgacaggactataaagataccaggcgtttccccctggaagctccctcgtgcgctctcctgttccg
accctgccgcttaccggatacctgtccgcctttctcccttcgggaagcgtggcgctttctcatagctcacgctgtaggtatctc.
pUC57 T7 promoter CSU 08 Toehold Switch TEV protease T7 terminator (T7
promoter, XbaI, CSU 08 Toehold Switch, Myc tag, TEV protease, HA tag, T7 terminator):
(SEQ ID NO: 45)
tcgcgcgtttcggtgatgacggtgaaaacctctgacacatgcagctcccggagacggtcacagcttgtctgtaagcggatgccggga
gcagacaagcccgtcagggcgcgtcagcgggtgttggcgggtgtcggggctggcttaactatgcggcatcagagcagattgtactg
agagtgcaccatatgcggtgtgaaataccgcacagatgcgtaaggagaaaataccgcatcaggcgccattcgccattcaggctgcgc
aactgttgggaagggcgatcggtgcgggcctcttcgctattacgccagctggcgaaagggggatgtgctgcaaggcgattaagttgg
gtaacgccagggttttcccagtcacgacgttgtaaaacgacggccagtgaattcgagctcggtacctcgcgaatgcatTAATAC
GACTCACTATAGGGTCTAGAGGGGCAGTTTCAAGAGTGCGGGAGAAAATTGATA
ACAGAGGAGAATCAATATGCTCCCGCACAACGGTAGCGCAGGTAGCGGTCATAT
GGAACAAAAGTTGATTTCTGAAGAAGACCTGGGAGAGTCATTATTCAAGGGTCC
ACGCGACTATAATCCTATCTCGTCTACAATCTGTCATCTGACTAACGAAAGCGAT
GGACATACTACGAGCCTTTACGGTATTGGCTTCGGGCCCTTTATTATTACTAATA
AGCACCTGTTCCGTCGTAATAATGGAACACTGCTTGTCCAGTCGTTGCATGGCGT
ATTCAAGGTGAAGAATACGACCACACTTCAACAGCACCTTATCGACGGTCGCGA
TATGATCATCATTCGCATGCCGAAAGATTTCCCACCATTTCCGCAGAAACTTAAG
TTCCGCGAACCACAGCGTGAGGAGCGCATCTGCCTGGTGACAACAAACTTTCAG
ACAAAGTCGATGTCAAGTATGGTCTCGGATACGAGCTGTACTTTTCCCTCTTCGG
ATGGCATTTTTTGGAAGCACTGGATTCAGACGAAGGACGGCCAATGTGGTTCGCC
TTTGGTCAGTACTCGTGACGGATTTATTGTGGGAATCCATAGCGCATCGAACTTT
ACAAACACTAATAACTATTTCACCTCTGTACCTAAAAACTTTATGGAACTGTTGA
CTAATCAGGAGGCTCAGCAATGGGTCAGTGGCTGGCGTCTTAACGCTGATAGCGT
ATTGTGGGGCGGGCACAAGGTATTTATGTCCAAACCCGAGGAGCCGTTCCAACC
AGTGAAAGAAGCGACACAACTGATGTCGGAGTTGGTATATTCCCAATACCCATA
CGATGTCCCAGACTATGCGTAATGAGGATCCCGGGAATTCTCGAGTAAGGTTAAC
CTGCAGGAGGCCTTTAATTAAGGTGGTGCGGCCGCGCTAGCGGTCCCGGGGGAT
CGATCCGGCTGCTAACAAAGCCCGAAAGGAAGCTGAGTTGGCTGCTGCCACCGC
TGAGCAATAACTAGCATAACCCCTTGGGGCCTCTAAACGGGTCTTGAGGGGTTTT
TTGatcggatcccgggcccgtcgactgcagaggcctgcatgcaagcttggcgtaatcatggtcatagctgtttcctgtgtgaaattgtt
atccgctcacaattccacacaacatacgagccggaagcataaagtgtaaagcctggggtgcctaatgagtgagctaactcacattaatt
gcgttgcgctcactgcccgctttccagtcgggaaacctgtcgtgccagctgcattaatgaatcggccaacgcgcggggagaggcggt
ttgcgtattgggcgctcttccgcttcctcgctcactgactcgctgcgctcggtcgttcggctgcggcgagcggtatcagctcactcaaag
gcggtaatacggttatccacagaatcaggggataacgcaggaaagaacatgtgagcaaaaggccagcaaaaggccaggaaccgta
aaaaggccgcgttgctggcgtttttccataggctccgcccccctgacgagcatcacaaaaatcgacgctcaagtcagaggtggcgaa
acccgacaggactataaagataccaggcgtttccccctggaagctccctcgtgcgctctcctgttccgaccctgccgcttaccggatac
ctgtccgcctttctcccttcgggaagcgtggcgctttctcatagctcacgctgtaggtatctcagttcggtgtaggtcgttcgctccaagct
gggctgtgtgcacgaaccccccgttcagcccgaccgctgcgccttatccggtaactatcgtcttgagtccaacccggtaagacacgac
ttatcgccactggcagcagccactggtaacaggattagcagagcgaggtatgtaggcggtgctacagagttcttgaagtggtggccta
actacggctacactagaagaacagtatttggtatctgcgctctgctgaagccagttaccttcggaaaaagagttggtagctcttgatccg
gcaaacaaaccaccgctggtagcggtggtttttttgtttgcaagcagcagattacgcgcagaaaaaaaggatctcaagaagatcctttg
atcttttctacggggtctgacgctcagtggaacgaaaactcacgttaagggattttggtcatgagattatcaaaaaggatcttcacctagat
ccttttaaattaaaaatgaagttttaaatcaatctaaagtatatatgagtaaacttggtctgacagttaccaatgcttaatcagtgaggcacct
atctcagcgatctgtctatttcgttcatccatagttgcctgactccccgtcgtgtagataactacgatacgggagggcttaccatctggccc
cagtgctgcaatgataccgcgagacccacgctcaccggctccagatttatcagcaataaaccagccagccggaagggccgagcgca
gaagtggtcctgcaactttatccgcctccatccagtctattaattgttgccgggaagctagagtaagtagttcgccagttaatagtttgcgc
aacgttgttgccattgctacaggcatcgtggtgtcacgctcgtcgtttggtatggcttcattcagctccggttcccaacgatcaaggcgag
ttacatgatcccccatgttgtgcaaaaaagcggttagctccttcggtcctccgatcgttgtcagaagtaagttggccgcagtgttatcactc
atggttatggcagcactgcataattctcttactgtcatgccatccgtaagatgcttttctgtgactggtgagtactcaaccaagtcattctga
gaatagtgtatgcggcgaccgagttgctcttgcccggcgtcaatacgggataataccgcgccacatagcagaactttaaaagtgctcat
cattggaaaacgttcttcggggcgaaaactctcaaggatcttaccgctgttgagatccagttcgatgtaacccactcgtgcacccaactg
atcttcagcatcttttactttcaccagcgtttctgggtgagcaaaaacaggaaggcaaaatgccgcaaaaaagggaataagggcgaca
cggaaatgttgaatactcatactcttcctttttcaatattattgaagcatttatcagggttattgtctcatgagcggatacatatttgaatgta
tttagaaaaataaacaaataggggttccgcgcacatttccccgaaaagtgccacctgacgtctaagaaaccattattatcatgacattaacct
ataaaaataggcgtatcacgaggccctttcgtc.
pUC57 SARS-Cov2 subfragment one(5’UTR, ORF1ab, Nsp2, Region 1, Region 2):
(SEQ ID NO: 46)
TTCCCAACGATCAAGGCGAGTTACATGATCCCCCATGTTGTGCAAAAAAGCGGTT
AGCTCCTTCGGTCCTCCGATCGTTGTCAGAAGTAAGTTGGCCGCAGTGTTATCAC
TCATGGTTATGGCAGCACTGCATAATTCTCTTACTGTCATGCCATCCGTAAGATG
CTTTTCTGTGACTGGTGAGTACTCAACCAAGTCATTCTGAGAATAGTGTATGCGG
CGACCGAGTTGCTCTTGCCCGGCGTCAATACGGGATAATACCGCGCCACATAGC
AGAACTTTAAAAGTGCTCATCATTGGAAAACGTTCTTCGGGGCGAAAACTCTCAA
GGATCTTACCGCTGTTGAGATCCAGTTCGATGTAACCCACTCGTGCACCCAACTG
ATCTTCAGCATCTTTTACTTTCACCAGCGTTTCTGGGTGAGCAAAAACAGGAAGG
CAAAATGCCGCAAAAAAGGGAATAAGGGCGACACGGAAATGTTGAATACTCATA
CTCTTCCTTTTTCAATATTATTGAAGCATTTATCAGGGTTATTGTCTCATGAGCGG
ATACATATTTGAATGTATTTAGAAAAATAAACAAATAGGGGTTCCGCGCACATTT
CCCCGAAAAGTGCCACCTGACGTCTAAGAAACCATTATTATCATGACATTAACCT
ATAAAAATAGGCGTATCACGAGGCCCTTTCGTCTCGCGCGTTTCGGTGATGACGG
TGAAAACCTCTGACACATGCAGCTCCCGGAGACGGTCACAGCTTGTCTGTAAGCG
GATGCCGGGAGCAGACAAGCCCGTCAGGGCGCGTCAGCGGGTGTTGGCGGGTGT
CGGGGCTGGCTTAACTATGCGGCATCAGAGCAGATTGTACTGAGAGTGCACCAT
ATGCGGTGTGAAATACCGCACAGATGCGTAAGGAGAAAATACCGCATCAGGCGC
CATTCGCCATTCAGGCTGCGCAACTGTTGGGAAGGGCGATCGGTGCGGGCCTCTT
CGCTATTACGCCAGCTGGCGAAAGGGGGATGTGCTGCAAGGCGATTAAGTTGGG
TAACGCCAGGGTTTTCCCAGTCACGACGTTGTAAAACGACGGCCAGTGAATTgacg
cgtattgggatattaaaggtttataccttcccaggtaacaaaccaaccaactttcgatctcttgtagatctgttctctaaacgaactttaaaat
ctgtgtggctgtcactcggctgcatgcttagtgcactcacgcagtataattaataactaattactgtcgttgacaggacacgagtaactcgt
ctatcttctgcaggctgcttacggtttcgtccgtgttgcagccgatcatcagcacatctaggtttcgtccgggtgtgaccgaaaggtaaga
tggagagccttgtccctggtttcaacgagaaaacacacgtccaactcagtttgcctgttttacaggttcgcgacgtgctcgtacgtggctt
tggagactccgtggaggaggtcttatcagaggcacgtcaacatcttaaagatggcacttgtggcttagtagaagttgaaaaaggcgtttt
gcctcaacttgaacagccctatgtgttcatcaaacgttcggatgctcgaactgcacctcatggtcatgttatggttgagctggtagcagaa
ctcgaaggcattcagtacggtcgtagtggtgagacacttggtgtccttgtccctcatgtgggcgaaataccagtggcttaccgcaaggtt
cttcttcgtaagaacggtaataaaggagctggtggccatagttacggcgccgatctaaagtcatttgacttaggcgacgagcttggcact
gatccttatgaagattttcaagaaaactggaacactaaacatagcagtggtgttacccgtgaactcatgcgtgagcttaacggaggggc
atacactcgctatgtcgataacaacttctgtggccctgatggctaccctcttgagtgcattaaagaccttctagcacgtgctggtaaagctt
catgcactttgtccgaacaactggactttattgacactaagaggggtgtatactgctgccgtgaacatgagcatgaaattgcttggtacac
ggaacgttctgaaaagagctatgaattgcagacaccttttgaaattaaattggcaaagaaatttgacaccttcaatggggaatgtccaaat
tttgtatttcccttaaattccataatcaagactattcaaccaagggttgaaaagaaaaagcttgatggctttatgggtagaattcgatctgtct
atccagttgcgtcaccaaatgaatgcaaccaaatgtgcctttcaactctcatgaagtgtgatcattgtggtgaaacttcatggcagacggg
cgattttgttaaagccacttgcgaattttgtggcactgagaatttgactaaagaaggtgccactacttgtggttacttaccccaaaatgctgt
tgttaaaatttattgtccagcatgtcacaattcagaagtaggacctgagcatagtcttgccgaataccataatgaatctggcttgaaaacca
ttcttcgtaagggtggtcgcactattgcctttggaggctgtgtgttctcttatgttggttgccataacaagtgtgcctattgggttccacgtgc
tagcgctaacataggttgtaaccatacaggtgttgttggagaaggttccgaaggtcttaatgacaaccttcttgaaatactccaaaaagag
aaagtcaacatcaatattgttggtgactttaaacttaatgaagagatcgccattattttggcatctttttctgcttccacaagtgcttttgtgg
aaactgtgaaaggtttggattataaagcattcaaacaaattgttgaatcctgtggtaattttaaagttacaaaaggaaaagctaaaaaaggtgc
ctggaatattggtgaacagaaatcaatactgagtcctctttatgcatttgcatcagaggctgctcgtgttgtacgatcaattttctcccgcac
tcttgaaactgctcaaaattctgtgcgtgttttacagaaggccgctataacaatactagatggaatttcacagtattcactgagactcattga
tgctatgatgttcacatctgatttggctactaacaatctagttgtaatggcctacattacaggtggtgttgttcagttgacttcgcagtggcta
actaacatctttggcactgtttatgaaaaactcaaacccgtccttgattggcttgaagagaagtttaaggaaggtgtagagtttcttagaga
cggttgggaaattgttaaatttatctcaacctgtgcttgtgaaattgtcggtggacaaattgtcacctgtgcaaaggaaattaaggagagtg
ttcagacattctttaagcttgtaaataaatttttggctttgtgtgctgactctatcattattggtggagctaaacttaaagccttgaatttagg
tgaaacatttgtcacgcactcaaagggattgtacagaaagtgtgttaaatccagagaagaaactggcctactcatgcctctaaaagcccca
aaagaaattatcttcttagagggagaaacacttcccacagaagtgttaacagaggaagttgtcttgaaaactggtgatttacaaccattag
aacaacctactagtgaagctgttgaagctccattggttggtacaccagtttgtattaacgggcttatgttgctcgaaatcaaagacacaga
aaagtactgtgcccttgcacctaatatgatggtaacaaacaataccttcacactcaaaggcggtgcaccaacaaaggttacttttggtgat
gacactgtgatagaagtgcaaggttacaagagtgtgaatatcacttttgaacttgatgaaaggattgataaagtacttaatgagaagtgct
ctgcctatacagttgaactcggtacagaagtaaatgagttcgcctgtgttgtggcagatgctgtcataaaaactttgcaaccagtatctga
attacttacaccactgggcattgatttagatgagtggagtatggctacatactacttatttgatgagtctggtgagtttaaattggcttcacat
atgtattgttctttctaccctccagatgaggatgaagaagaaggtgattgtgaagaagaagagtttgagccatcaactcaatatgagtatg
gtactgaagatgattaccaaggtaaacctttggaatttggtgccacttctgctgctcttcaacctgaagaagagcaagaagaagattggtt
agatgatgatagtcaacaaactgttggtcaacaagacggcagtgaggacaatcagacaactactattcaaacaattgttgaggttcaac
ctcaattagagatggaacttacaccagttgttcagactattgaagtgaatagttttagtggttatttaaaacttactgacaatgtatacatta
aaaatgcagacattgtggaagaagctaaaaaggtaaaaccaacagtggttgttaatgcagccaatgtttaccttaaacatggaggaggtgtt
gcaggagccttaaataaggctactaacaatgccatgcaagttgaatctgatgattacatagctactaatggaccacttaaagtgggtggt
agttgtgttttaagcggacacaatcttgctaaacactgtcttcatgttgtcggcccaaatgttaacaaaggtgaagacattcaacttcttaag
agtgcttatgaaaattttaatcagcacgaagttctacttgcaccattattatcagctggtatttttggtgctgaccctatacattctttaagag
tttgtgtagatactgttcgcacaaatgtctacttagctgtctttgataaaaatctctatgacaaacttgtttcaagctttttggaaatgaaga
gtgaaaagcaagttgaacaaaagatcgctgagattcctaaagaggaagttaagccatttataactgaaagtaaaccttcagttgaacagagaa
aacaagatgataagaaaatcaaagcttgtgttgaagaagttacaacaactctggaagaaactaagttcctcacagaaaacttgttacttta
tattgacattaatggcaatcttcatccagattctgccactcttgttagtgacattgacatcactttcttaaagaaagatgctccatatatagtgg
gtgatgttgttcaagagggtgttttaactgctgtggttatacctactaaaaaggctggtggcactactgaaatgctagcgaaagctttgag
aaaagtgccaacagacaattatataaccacttacccgggtcagggtttaaatggttacactgtagaggaggcaaagacagtgcttaaaa
agtgtaaaagtgccttttacattctaccatctattatctctaatgagaagcaagaaattcttggaactgtttcttggaatttgcgagaaatgctt
gcacatgcagaagaaacacgcaaattaatgcctgtctgtgtggaaactaaagccatagtttcaactatacagcgtaaatataagggtatt
aaaatacaagagggtgtggttgattatggtgctagattttacttttacaccagtaaaacaactgtagcgtcacttatcaacacacttaacgat
ctaaatgaaactcttgttacaatgccacttggctatgtaacacatggcttaaatttggaagaagctgctcggtatatgagatctctcaaagt
gccagctacagtttctgtttcttcacctgatgctgttacagcgtataatggttatcttacttcttcttctaaaacacctgaagaacattttatt
gaaaccatctcacttgctggttcctataaagattggtcctattctggacaatctacacaactaggtatagaatttcttaagagaggtgataaaag
tgtatattacactagtaatcctaccacattccacctagatggtgaagttatcacctttgacaatcttaagacacttctttctttgagagaagtg
aggactattaaggtgtttacaacagtagacaacattaacctccacacgcaagttgtggacatgtcaatgacatatggacaacagtttggt
ccaacttatttggatggagctgatgttactaaaataaaacctcataattcacatgaaggtaaaacattttatgttttacctaatgatgacactct
acgtgttgaggcttttgagtactaccacacaactgatcctagttttctgggtaggtacatgtcagcattaaatcacactaaaaagtggaaat
acccacaagttaatggtttaacttctattaaatgggcagataacaactgttatcttgccactgcattgttaacactccaacaaatagagttga
agtttaatccacctgctctacaagatgcttattacagagcaagggctggtgaagctgctaacttttgtgcacttatcttagcctactgtaata
agacagtaggtgagttaggtgatgttagagaaacaatgagttacttgtttcaacatgccaatttagattcttgcaaaagagtcttgaacgtg
gtgtgtaaaacttgtggacaacagcagacaacccttaagggtgtagaagctgttatgtacatgggcacactttcttatgaacaatttaaga
aaggtgttcagataccttgtacgtgtggtaaacaagctacaaaatatctagtacaacaggagtcaccttttgttatgatgtcagcaccacct
gctcagtatgaacttaagcatggtacatttacttgtgctagtgagtacactggtaattaccagtgtggtcactataaacatataacttctaaa
gaaactttgtattgcatagacggtgctttacttacaaagtcctcagaatacaaaggtcctattacggatgttttctacaaagaaaacagttac
acaacaaccataaaaccagttacttataaattggatggtgttgtttgtacagaaattgaccctaagttggacaattattataagaaagacaa
ttcttatttcacagagcaaccaattgatcttgtaccaaaccaaccatatccaaacgcaagcttcgataattttaagtttgtatgtgataatatc
aaatttgctgatgatttaaaccagttaactggttataagaaacctgcttcaagagagcttaaagttacatttttccctgacttaaatggtgatg
tggtggctattgattataaacactacacaccctcttttaagaaaggagctaaattgttacataaacctattgtttggcatgttaacaatgcaac
taataaagccacgtataaaccaaatacctggtgtatacgttgtctttggagcacaaaaccagttgaaacatcaaattcgtttgatgtactga
agtcagaggacgcgcagggaatggataatcttgcctgcgaagatctaaaaccagtctctgaagaagtagtggaaaatcctaccataca
gaaagacgttcttgagtgtaatgtgaaaactaccgaagttgtaggagacattatacttaaaccagcaaataatagtttaaaaattacagaa
gaggttggccacacagatctaatggctgcttatgtagacaattctagtcttactattaagaaacctaatgaattatctagagtattaggtttg
aaaacccttgctactcatggtttagctgctgttaatagtgtcccttgggatactatagctaattatgctaagccttttcttaacaaagttgttag
tacaactactaacatagttacacggtgtttaaaccgtgtttgtactaattatatgccttatttctttactttattgctacaattgtgtactttt
actagaagtacaaattctagaatAATTCGAGCTCGGTACCTCGCGAATGCATCTAGATATCGGATCC
CGGGCCCGTCGACTGCAGAGGCCTGCATGCAAGCTTGGCGTAATCATGGTCATA
gctgtttcctgtgtgaaattgttatccgctcacaattccacacaacatacgagcc
GGAAGCATAAAGTGTAAAGCCTGGGGTGCCTAATGAGTGAGCTAACTCACATTA
ATTGCGTTGCGCTCACTGCCCGCTTTCCAGTCGGGAAACCTGTCGTGCCAGCTGC
ATTAATGAATCGGCCAACGCGCGGGGAGAGGCGGTTTGCGTATTGGGCGCTCTTC
CGCTTCCTCGCTCACTGACTCGCTGCGCTCGGTCGTTCGGCTGCGGCGAGCGGTA
TCAGCTCACTCAAAGGCGGTAATACGGTTATCCACAGAATCAGGGGATAACGCA
GGAAAGAACATGTGAGCAAAAGGCCAGCAAAAGGCCAGGAACCGTAAAAAGGC
CGCGTTGCTGGCGTTTTTCCATAGGCTCCGCCCCCCTGACGAGCATCACAAAAAT
CGACGCTCAAGTCAGAGGTGGCGAAACCCGACAGGACTATAAAGATACCAGGCG
TTTCCCCCTGGAAGCTCCCTCGTGCGCTCTCCTGTTCCGACCCTGCCGCTTACCGG
ATACCTGTCCGCCTTTCTCCCTTCGGGAAGCGTGGCGCTTTCTCATAGCTCACGCT
GTAGGTATCTCAGTTCGGTGTAGGTCGTTCGCTCCAAGCTGGGCTGTGTGCACGA
ACCCCCCGTTCAGCCCGACCGCTGCGCCTTATCCGGTAACTATCGTCTTGAGTCC
AACCCGGTAAGACACGACTTATCGCCACTGGCAGCAGCCACTGGTAACAGGATT
AGCAGAGCGAGGTATGTAGGCGGTGCTACAGAGTTCTTGAAGTGGTGGCCTAAC
TACGGCTACACTAGAAGAACAGTATTTGGTATCTGCGCTCTGCTGAAGCCAGTTA
CCTTCGGAAAAAGAGTTGGTAGCTCTTGATCCGGCAAACAAACCACCGCTGGTA
GCGGTGGTTTTTTTGTTTGCAAGCAGCAGATTACGCGCAGAAAAAAAGGATCTCA
AGAAGATCCTTTGATCTTTTCTACGGGGTCTGACGCTCAGTGGAACGAAAACTCA
CGTTAAGGGATTTTGGTCATGAGATTATCAAAAAGGATCTTCACCTAGATCCTTT
TAAATTAAAAATGAAGTTTTAAATCAATCTAAAGTATATATGAGTAAACTTGGTC
TGACAGTTACCAATGCTTAATCAGTGAGGCACCTATCTCAGCGATCTGTCTATTT
CGTTCATCCATAGTTGCCTGACTCCCCGTCGTGTAGATAACTACGATACGGGAGG
GCTTACCATCTGGCCCCAGTGCTGCAATGATACCGCGAGACCCACGCTCACCGGC
TCCAGATTTATCAGCAATAAACCAGCCAGCCGGAAGGGCCGAGCGCAGAAGTGG
TCCTGCAACTTTATCCGCCTCCATCCAGTCTATTAATTGTTGCCGGGAAGCTAGA
GTAAGTAGTTCGCCAGTTAATAGTTTGCGCAACGTTGTTGCCATTGCTACAGGCA
TCGTGGTGTCACGCTCGTCGTTTGGTATGGCTTCATTCAGCTCCGG.

### 4. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Selection of SARS-CoV-2 Genome Regions for Designing Toehold Sensor Switches. A combination of strategies was used for optimal design of the toehold switches. This included identifying regions that were: 1) conserved among SARS-CoV-2 isolates, 2) unique to SARS-CoV-2 but not to common low pathogenic human coronaviruses, 3) contained minimal secondary structure that could potentially interfere with viral RNA binding to the toehold switch, and 4) not prone to mutations that could interrupt viral RNA binding.

Four conserved regions in the SARS-CoV-2 genome (nucleotide regions 1,865-2,018, 21,731-21,788, 23,536-23,598, and 27,977-28,909) were previously reported as unique to SARS-CoV-2 when compared with other coronaviruses. A study by Rangan et al. found that secondary structures were absent in the nt region 1,918-1,933. The mutation rate of this region encoding nonstructural protein 2 (nsp2) was found to be below 5% in North America, Asia, and European strains of SARS-CoV-2.

Based on these considerations, experiments focused on designing toeholds to bind the SARS-CoV-2 genome between nt positions 1,918-1,933 (TCTTGAAACTGCTCAA) designated region 1 and was expanded to include nt positions 1,865-2,018 designated region 2 (SARS-CoV-2 Wuhan strain; NCBI Reference Sequence NC_045512.2). The rationale was that this region was conserved among SARS-CoV-2 isolates but unique from other common coronaviruses, there was little secondary structure, and this region would likely show less variation since it encoded a nonstructural protein (nsp2) and therefore would be under less selective pressure for mutation hotspots due to vaccine usage and infections.

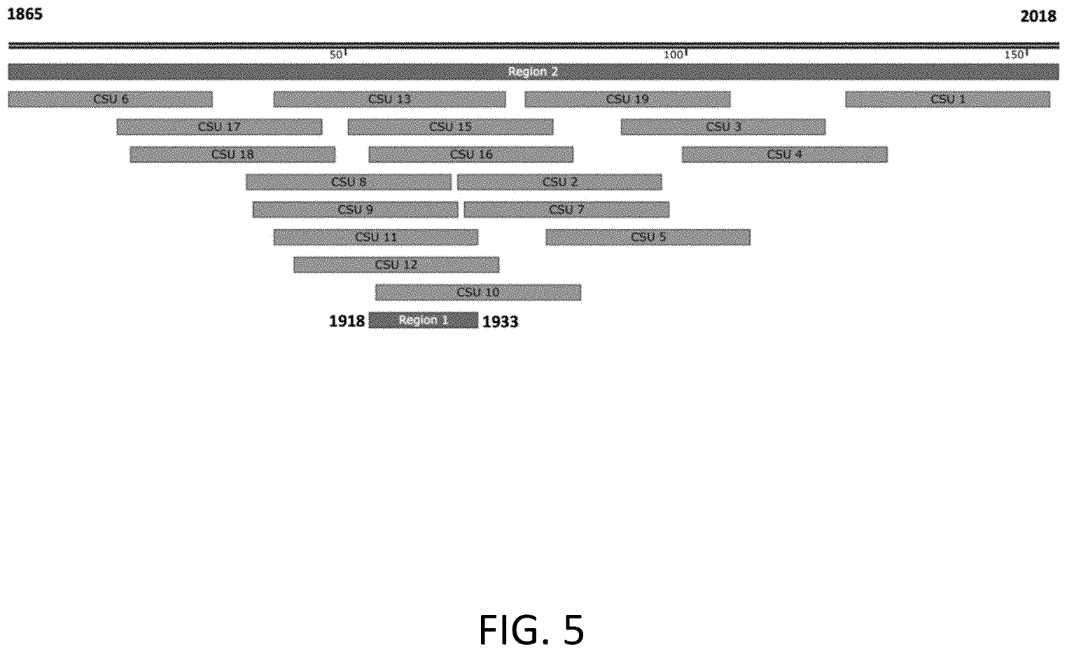

After determining the optimal target SARS-CoV-2 genome region, switches were designed according to criteria used in the previous studies of Green et al. The location of the inducer RNA for each toehold switch design is shown on the 1,865-2,018 nt region of the SARS-CoV-2 genome (FIG. 5).

### Example 2

In Silico Evaluation of Toehold Switches. The free energy of folding (AG) and the conformation of the folded toehold switch sequences were measured using the RNAFold and NUPACK software packages, respectively (rna.tbi.univie.ac.at/cgi-bin/RNAWebSuite/RNAfold.cgi; nupack.org). The predicted on/off ratios for the toehold switch designs were evaluated with STORM (Sequence-based Toehold Optimization & Redesign Model) software and the designs were ranked according to the predicted on/off ratios. The on/off ratios reflect the efficiency of hybridization between the inducer and toehold switch with the ‘on’ and ‘off’ state correlating to the toehold switch plus inducer and toehold switch minus inducer (storm-toehold.herokuapp.com). The sequence of the toehold switches and their on/off ratios are shown in Table 1 below.

### Example 3

Screening of Toeholds and Inducers. The top 19 ranking toehold switch designs, as determined by the STORM software, were tested along with their complementary inducers. Since ranking by the STORM software alone may not necessarily identify the best functioning toehold switch, the toehold-inducer combinations were empirically tested in a commercially available cell-free protein synthesis (CFPS) system for production of mNeonGreen fluorescent protein. In initial experiments, the toehold switch-plasmid was mixed with the cell extract based-CFPS system with or without the respective RNA inducer-plasmid, and the resulting fluorescence was measured over time. A fold-change calculation comparing the ratios of the slope of the fluorescent signal change in the induced and uninduced reactions was used to determine the significance of the on/off states of the toehold switches and to identify which toehold switches gave the highest reporter signal compared to background noise.

Toehold switch sensors (CSU 01, 04, and 08) showed a high on/off fold-change, that is, high relative fluorescence at the on-state but low relative fluorescence at the off-state and were selected for further experiments. The toehold switches with a predicted on/off ratio greater than 0.3 showed the best signaling.

Toehold switch CSU 08 gave the most consistent, and largest, fold-changes in fluorescence, and subsequent efforts focused on this switch to save time and resources (FIG. 6). The CSU 08 inducer RNA also corresponds to the region at SARS Cov-2 nucleotides 1,918-1,933 (region 1) which was the region that best fit all of the toehold switch design criteria.

### Example 4

Limit of detection with small and large inducer RNA and mNeonGreen reporter. After initial testing of a series of inducer RNA concentrations from 5000 nM to 1 nM, it was found that signaling from toehold switch CSU 08 was initiated by as little as 300 nM of inducer RNA (data not shown). Unfortunately, these results led to the conclusion that the sensor would be unsuccessful in the detection of RNA in clinical samples, considering that the average viral load of SARS-CoV-2 is 7.99×10⁴ copies/mL (0.13 fM) in an upper respiratory tract sample and 7.52×10⁵ copies/mL (1.25 fM) in a lower respiratory tract sample.

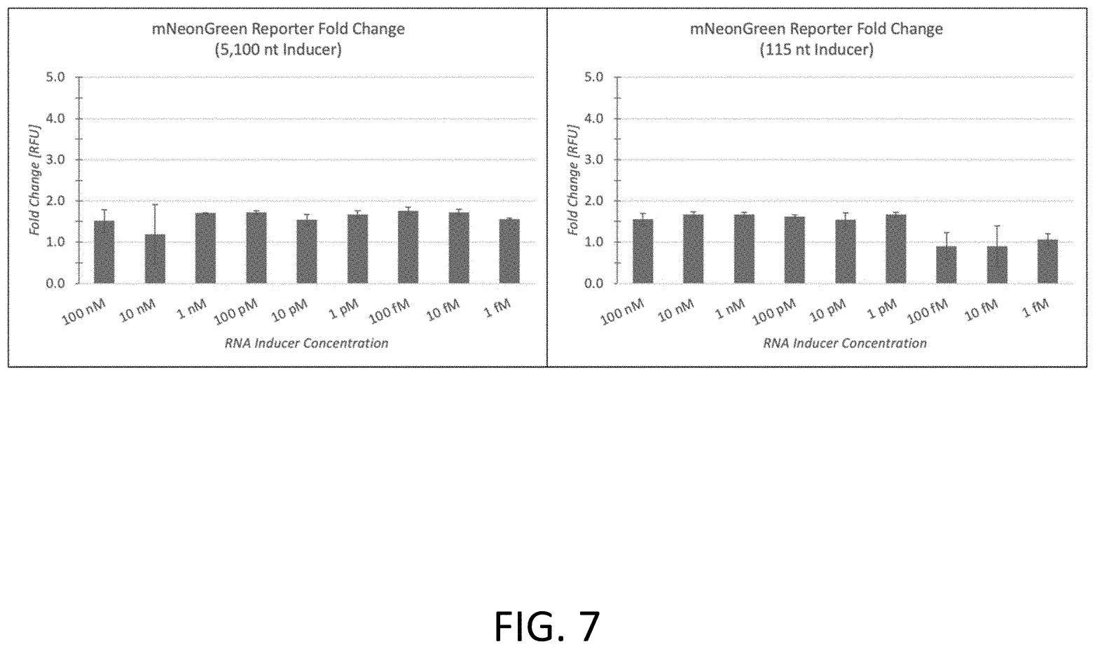

A CFPS system (NEBExpress) which contained cell lysate was used in the preliminary limit of detection experiments to lower experimental costs. However, the cell lysate contributed a higher fluorescent background which interfered with the determination of the lower detection limits. Therefore, in subsequent experiments, a more purified, defined CFPS system (NEB PURExpress) was used to minimize background fluorescence and increase the sensitivity of detection. To see if it was possible to detect concentrations below 300 nM, an additional range of inducer RNA with concentrations spanning from 100 nM to 1 fM was tested with the CSU 08 toehold switch and the defined CFPS system. The lowest RNA concentration that achieved some signaling (−1.5 fold) above background was found to be 1 pM (FIG. 7). This signaling is still above the sensitivity threshold needed to create a robust diagnostic.

To test the potential for toehold switch CSU 08 to function in the presence of larger RNA sequences, RNA was initially produced for the first 6.8 kb of SARS-CoV-2 genome, which contains inducer regions 1 and 2. However, RNA of approximately 1.5 kb was produced using a commercial T7 RNA synthesis kit. Subsequently, it was discovered that there was potentially a cryptic T7 terminator that stopped transcription; therefore, the upper primer was moved to start amplification downstream of the cryptic terminator. Both the reaction conditions and reagents were modified to obtain a long RNA transcript because commercial RNA synthesis kits are optimized for producing shorter transcripts. Thus a ca. 5,100 nt fragment (nucleotides 1,729 to 6,834 of the SARS-CoV-2 genome) flanked by a T7 promoter and terminator was amplified from a subgenomic SARS-CoV-2 cDNA. This subgenomic fragment contained the RNA inducer region for the toehold switches and was used to generate 5,100 nt inducer RNA. Fold changes for 5,100 nucleotides were comparable to the small RNA inducer (FIG. 7). Signal was detected in the fM inducer RNA range with the 5,100 nt inducer whereas signal detection with the 115 nt inducer went to 1 pM.

### Example 5

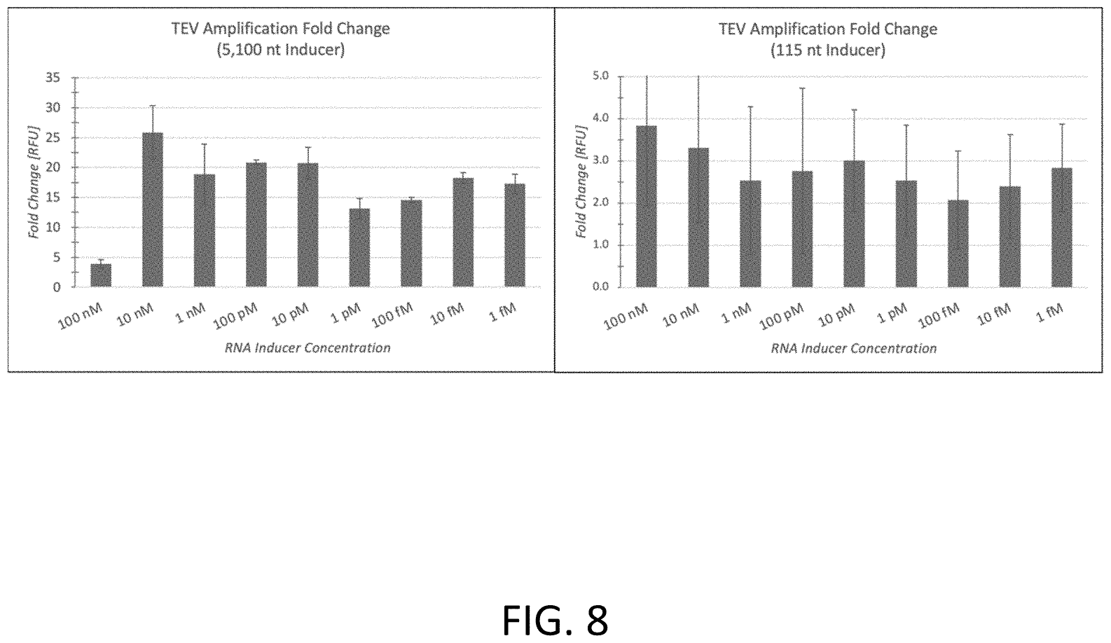

TEV Assay Limit of Detection. In an effort to lower the limit of detection, a signal amplification consisting of a new reporter system, using the Tobacco Etch Virus (TEV) protease gene, was engineered downstream of toehold switch CSU 08 in order to proteolytically activate a quenched 5-carboxyfluorescein (5-Fam dye) reporter substrate. An E. coli codon optimized version of TEV protease, with a 5′ UTR CSU 08 toehold switch and flanked by a T7 promoter and T7 terminator was synthesized by Synbio Technologies in a pUC57 plasmid. As in the mNeonGreen reporter limit of detection experiments, the 115 or 5,100 nt inducers were tested over a 100 nM to 1 fM concentration range. Upon binding of the inducer to CSU 08 and translation of TEV protease, the QXL 520 quencher-TEV cleavage site-5-FAM dye substrate complex was cleaved releasing the fluorescent 5-FAM dye. The highest fold change in fluorescence for the 115 nt RNA inducer occurred in the 100 nM reactions at 3.84 with standard deviation of 1.90 when compared to the uninduced wells. The lowest fold change for the 115 nt RNA inducer occurred in the 100 fM reactions at 2.08 with standard deviation of 1.16. Signal was detected at the lowest inducer concentration tested, 1 fM. The fold change range of 2 to 4-fold seen using the amplification system and the 115 nt inducer was much better than what was seen with just the 115 nt inducer and the mNeonGreen reporter alone which showed a fold change range between 1 to 2-fold (FIG. 8).

The highest fold change for the 5,100 nt RNA inducer occurred in the reactions with 10 nM final concentration of RNA inducer, at an average 22.79 fold change with a standard deviation of 3.92. The lowest fold change for the 5,100 nt RNA inducer occurred in the reactions with 100 nM final concentration of RNA inducer, at an average of 3.56 and a standard deviation of 0.63. The uncharacteristically low reading at the highest concentration of the RNA inducer may be due to overcrowding in the reaction and steric inhibition. It has been reported that macromolecular crowding can have an inhibitory effect on translation in cell free systems. The 5,100 nt RNA inducer showed sensitivity down to 1 fM, indicating that the lower limit of detection for the TEV enzyme amplification toehold sensor has not yet been reached. The 1 fM concentration of the 5,100 nt RNA induced reactions showed an average fold change of 15.29 and standard deviation of 1.42 when compared to the uninduced wells. These results outperform the mNeonGreen reporter by ˜15 fold at 1 fm and are in the realm of viral load in human clinical samples. The 5,100 nt SARS-COV-2 subgenomic RNA inducer outperformed the 115 nt RNA inducer in the TEV amplification assays (FIG. 8).

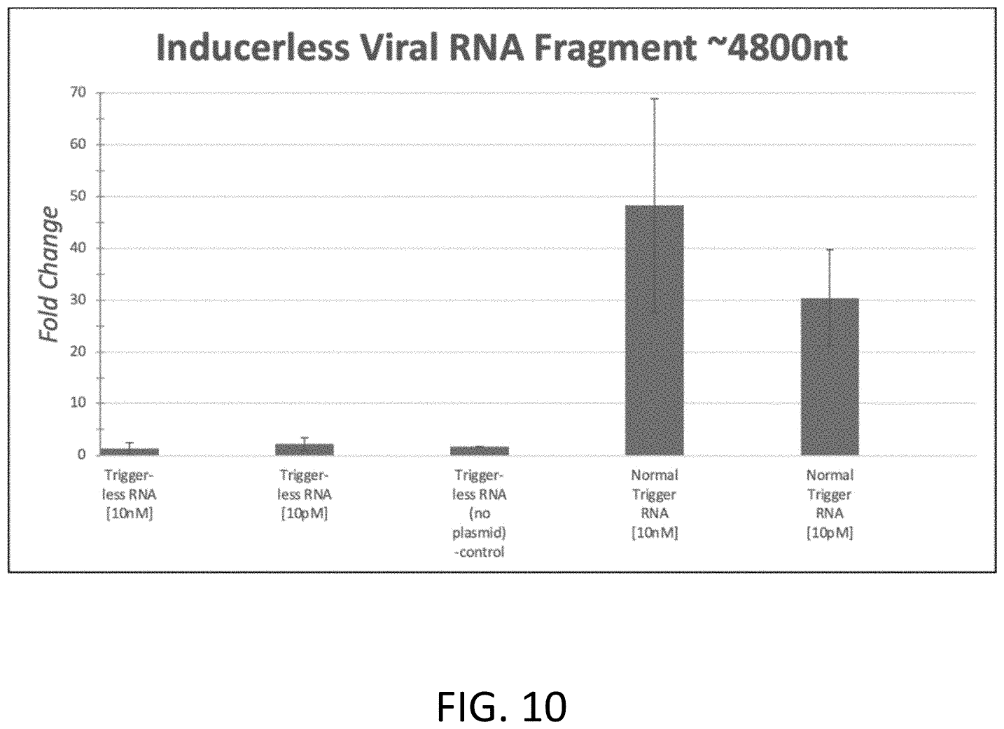

As described further herein, the use of the mNeonGreen reporter allowed detection of less than 1.5×10⁷ copies of the 115 nt inducer RNA, if a cut-off of a 1.5-fold change was used. The TEV protease amplification system detected less than 15,000 copies of the 115 nt inducer RNA and gave a 1.7-fold higher change in relative fluorescence compared with the mNeonGreen reporter at the lowest limit of detection (1 pM) for the mNeonGreen reporter. In contrast, the 5,100 nt RNA fragment was more efficient in inducing detection in the TEV protease amplification system and the limit of detection was less than 15,000 copies with an approximate 10-fold change in relative fluorescence over the mNeonGreen reporter. Even in the mNeonGreen reporter system, the limit of detection was lowered from a low pM range to a fM range when the 5,100 nt RNA fragment was used as the inducer. This superior performance with the larger RNA fragment was somewhat unexpected and was not due to a nonspecific effect of the 5100 nt inducer fragment affecting toehold switch activation since inclusion of a control RNA fragment lacking the CSU 08 inducer sequence but containing flanking SARS-CoV-2 regions did not cause an increase in relative fluorescence (FIG. 10).

What is claimed is:

1. A composition for performing an analyte detection assay, the composition comprising:
a detection component comprising an RNA molecule capable of binding an analyte, wherein the RNA molecule comprises an analyte recognition domain, a ribosome binding site, and an initiator protein domain; and
a signal amplification component responsive to expression of an initiator protein encoded by the initiator protein domain;
wherein presence of the analyte causes expression of the initiator protein and subsequent activation of the signal amplification component.

2. The composition of claim 1, wherein the RNA molecule forms a hairpin structure, wherein the hairpin structure is disrupted upon binding of the analyte to the analyte recognition domain, thereby exposing ribosome binding site and the initiator protein domain.

3. The composition of claim 1, wherein the initiator protein comprises an enzyme.

4. The composition of claim 3, wherein the enzyme is adenylate cyclase, or a derivative or variant thereof.

5. The composition of claim 4, wherein the adenylate cyclase, or a derivative or variant thereof, is from a bacterium, a virus, an archaeon, a fungus, a protozoan, a vertebrate, a non-vertebrate, or a plant.

6. The composition of claim 4, wherein the adenylate cyclase, or a derivative or variant thereof, is from Bordetella pertussis.

7. The composition of claim 3, wherein the enzyme is a protease, or a derivative or variant thereof.

8. The composition of claim 7, wherein the protease, or a derivative or variant thereof, is from a bacterium, a virus, an archaeon, a fungus, a protozoan, a vertebrate, a non-vertebrate, or a plant.

9. The composition of claim 7, wherein the protease, or a derivative or variant thereof, is a plant virus protease.

10. The composition of claim 9, wherein the plant virus protease is a tobacco etch virus (TEV) protease, a papain-like cystine protease, or a glutamic protease.

11. The composition of claim 1, wherein the signal amplification component comprises:
(i) a peptide comprising a protease cleavage site coupled to a fluorescent molecule and a quencher molecule, wherein the fluorescent molecule is activated upon cleavage of the protease cleavage cite by a protease and/or a cascade of proteases;
(ii) a luciferase complex comprising luciferin, or a derivative or variant thereof, and a luciferase or split luciferase comprising a protease cleavage site, wherein the luciferase or split luciferase is activated upon cleavage of the protease cleavage site by a protease and/or a cascade of proteases;
(iii) a fluorescent protein or polypeptide comprising a protease cleavage site, wherein the fluorescent protein or polypeptide is activated upon cleavage of the protease cleavage site by a protease and/or a cascade of proteases;
(iv) one or more components of a glycogenolysis complex;
(v) one or more components of a beta-galactosidase complex; or
(vi) one or more nucleic acid aptamers.

12. The composition of claim 11, wherein the peptide comprises a TEV-specific protease cleavage site comprising ENLYFQG (SEQ ID NO: 42), wherein the fluorescent molecule is a 5-FAM dye, and wherein the quencher is a QLX 520 quencher.

13. The composition of claim 11, wherein the one or more components of a glycogenolysis complex comprises: (i) Protein Kinase A, Phosphorylase Kinase b, and Glycogen Phosphorylase, or phosphorylated derivatives thereof; and (ii) cAMP, ADP, and glucose-1-P.

14. The composition of claim 11, wherein the fluorescent protein or polypeptide comprises GFP, mNeonGreen, YFP, RFP, or CFP, or a variant or derivative thereof.

15. The composition of claim 11, wherein the one or more components of a beta-galactosidase complex comprises: (i) 5-bromo-4-chloro-3-indolyl-β-D-galactopyranoside (X-gal), ortho-Nitrophenyl-ß-galactoside (ONPG), 6-chloro-3-indolyl-β-D-galactopyranoside (S-gal), 5-bromo-6-chloro-3-indolyl-β-D-galactopyranoside (Magenta-gal), 5-bromo-3-indolyl-β-D-galactopyranoside (Bluo-gal), p-nitrophenyl-β-d-galactopyranoside (PNPG); and (ii) beta-galactosidase, or a derivative or variant thereof.

16. The composition of claim 11, wherein the one or more nucleic acid aptamers comprises a Broccoli 3WJdB aptamer, and wherein the signal amplification component further comprises a DFHBI-1T dye.

17. The composition of claim 1, wherein the composition comprises one or more components of a cell free protein expression system.

18. The composition of claim 1, wherein the composition comprises a reaction buffer.

19. The composition of claim 1, wherein the analyte comprises at least one of a DNA molecule, an RNA molecule, a small molecule, a lipid, a peptide, a polypeptide, a protein, or a glycoprotein.

20. The composition of claim 1, wherein the analyte is from a pathogenic organism selected from the group consisting of bacteria, viruses, protozoa, worms, and fungi.

21. The composition of claim 1, wherein the analyte comprises RNA from a virus.

22. The composition of claim 21, wherein the virus is a SARS-CoV-2.

23. The composition of claim 1, wherein the RNA molecule comprises the nucleic acid sequence:
GGGNNNNNNNNNNNNNNNNNNNNNNNNNNNNNNAACAGAGGAGANNNNNNAU GNNNNNNNNNAACGGUAGCGCAGGUAGCGGCAUAUG (SEQ ID NO: 1).

24. The composition of claim 1, wherein the RNA molecule comprises a nucleic acid sequence selected from the group consisting of SEQ ID NOs: 2-19.

25. A method for detecting an analyte, the method comprising:
combining the composition comprising the detection component and signal amplification component of claim 1 with a biological sample; and
measuring or detecting a signal produced by the signal amplification component;
wherein presence of the analyte in the biological sample causes expression of an initiator protein and subsequent activation of the signal amplification component.

26. The method of claim 25, wherein the biological sample comprises a blood sample, a plasma sample, a serum, sample, a cerebral spinal fluid sample, a saliva sample, a tear sample, a urine sample, a fecal sample, a cell sample, a tissue sample, a water sample, and/or a plant sample.

27. The method of claim 25, wherein the signal is a fluorescent signal, a bioluminescent signal, a chemical signal, an electrochemical signal, or a colorimetric signal.

28. The method of claim 25, wherein the method further comprises quantifying the signal and determining a concentration of the analyte in the biological sample.

29. The method of claim 25, wherein the analyte is present in the sample at a concentration ranging from about 1 fM to about 1 μM.

30. The method of claim 25, wherein the analyte comprises viral RNA from SARS-CoV-2.

31. A kit comprising the composition of claim 1 and instructions for performing an analyte detection assay.

32. The kit of claim 31, wherein the kit comprises one or more components of a cell free protein expression system and/or a reaction buffer.

33. A composition for performing an analyte detection assay, the composition comprising:
a detection component comprising an allosteric transcription factor comprising an analyte recognition domain, and a RNA molecule comprising an allosteric transcription factor binding domain, a ribosome binding site, and an initiator protein domain; and
a signal amplification component responsive to expression of an initiator protein encoded by the initiator protein domain;
wherein presence of the analyte causes expression of the initiator protein and subsequent activation of the signal amplification component.

34. The composition of claim 33, wherein the allosteric transcription factor is selected from the group consisting of:
(i) a TetR transcription factor selected from TetR, MphR, QacR, and TtgR, and any variants or derivates thereof;
(ii) a multiple antibiotic resistance repressor (MarR) transcription factors selected from OtrR, CtcS, MobR, and HucR, and any variants or derivates thereof;
(iii) an ArsR/SmtB transcriptional regulator selected from SmtB and CadC, and any variants or derivates thereof;
(iv) a CsoR/RcnR transcriptional regulator selected from CsoR, and any variants or derivates thereof;
(v) a MerR transcriptional regulator, and any variants or derivates thereof;
(vi) a Fur transcriptional regulator, and any variants or derivates thereof;
(vii) a DtxR transcriptional regulator, and any variants or derivates thereof;
(viii) a NikR transcriptional regulator, and any variants or derivates thereof; and
(ix) a xylR transcriptional activator acting on toluene, xylene, benzene, and any variants or derivatives thereof.

35. The composition of claim 33, wherein the initiator protein comprises an enzyme.

36. The composition of claim 35, wherein the enzyme is adenylate cyclase, or a derivative or variant thereof.

37. The composition of claim 36, wherein the adenylate cyclase, or a derivative or variant thereof, is from a bacterium, a virus, an archaeon, a fungus, a protozoan, a vertebrate, a non-vertebrate, or a plant.

38. The composition of claim 36, wherein the adenylate cyclase, or a derivative or variant thereof, is from Bordetella pertussis.

39. The composition of claim 35, wherein the enzyme is a protease, or a derivative or variant thereof.

40. The composition of claim 39, wherein the protease, or a derivative or variant thereof, is from a bacterium, a virus, an archaeon, a fungus, a protozoan, a vertebrate, a non-vertebrate, or a plant.

41. The composition of claim 39, wherein the protease, or a derivative or variant thereof, is a plant virus protease.

42. The composition of claim 41, wherein the plant virus protease is a tobacco etch virus (TEV) protease, a papain-like cystine protease, or a glutamic protease.

43. The composition of claim 33, wherein the signal amplification component comprises:
(i) a peptide comprising a protease cleavage site coupled to a fluorescent molecule and a quencher molecule, wherein the fluorescent molecule is activated upon cleavage of the protease cleavage cite by a protease and/or a cascade of proteases;
(ii) a luciferase complex comprising luciferin, or a derivative or variant thereof, and a luciferase or split luciferase comprising a protease cleavage site, wherein the luciferase or split luciferase is activated upon cleavage of the protease cleavage site by a protease and/or a cascade of proteases;
(iii) a fluorescent protein or polypeptide comprising a protease cleavage site, wherein the fluorescent protein or polypeptide is activated upon cleavage of the protease cleavage site by a protease and/or a cascade of proteases;
(iv) one or more components of a glycogenolysis complex;
(v) one or more components of a beta-galactosidase complex; or
(vi) one or more nucleic acid aptamers.

44. The composition of claim 43, wherein the peptide comprises a TEV-specific protease cleavage site comprising ENLYFQG (SEQ ID NO: 42), wherein the fluorescent molecule is a 5-FAM dye, and wherein the quencher is a QLX 520 quencher.

45. The composition of claim 43, wherein the one or more components of a glycogenolysis complex comprises: (i) Protein Kinase A, Phosphorylase Kinase b, and Glycogen Phosphorylase, or phosphorylated derivatives thereof; and (ii) cAMP, ADP, and glucose-1-P.

46. The composition of claim 43, wherein the fluorescent protein or polypeptide comprises GFP, mNeonGreen, YFP, RFP, or CFP, or a variant or derivative thereof.

47. The composition of claim 43, wherein the one or more components of a beta-galactosidase complex comprises: (i) 5-bromo-4-chloro-3-indolyl-β-D-galactopyranoside (X-gal), ortho-Nitrophenyl-ß-galactoside (ONPG), 6-chloro-3-indolyl-β-D-galactopyranoside (S-gal), 5-bromo-6-chloro-3-indolyl-β-D-galactopyranoside (Magenta-gal), 5-bromo-3-indolyl-β-D-galactopyranoside (Bluo-gal), p-nitrophenyl-β-d-galactopyranoside (PNPG); and (ii) beta-galactosidase, or a derivative or variant thereof.

48. The composition of claim 43, wherein the one or more nucleic acid aptamers comprises a Broccoli 3WJdB aptamer, and wherein the signal amplification component further comprises a DFHBI-1T dye.

49. The composition of claim 33, wherein the composition comprises one or more components of a cell free protein expression system.

50. The composition of claim 33, wherein the composition comprises a reaction buffer.

50. The composition of claim 33, wherein the analyte comprises at least one of a DNA molecule, an RNA molecule, a small molecule, a lipid, a peptide, a polypeptide, a protein, or a glycoprotein.

51. A method for detecting an analyte, the method comprising:
combining the composition comprising the detection component and signal amplification component of claim 33 with a biological sample; and
measuring or detecting a signal produced by the signal amplification component;
wherein presence of the analyte in the biological sample causes expression of an initiator protein and subsequent activation of the signal amplification component.

52. The method of claim 51, wherein the biological sample comprises a blood sample, a plasma sample, a serum, sample, a cerebral spinal fluid sample, a saliva sample, a tear sample, a urine sample, a fecal sample, a cell sample, a tissue sample, a water sample, and/or a plant sample.

53. The method of claim 51, wherein the signal is a fluorescent signal, a bioluminescent signal, a chemical signal, an electrochemical signal, or a colorimetric signal.

54. The method of claim 51, wherein the method further comprises quantifying the signal and determining a concentration of the analyte in the biological sample.

55. The method of claim 51, wherein the analyte is present in the sample at a concentration ranging from about 1 fM to about 1 μM.

56. A kit comprising the composition of claim 33 and instructions for performing an analyte detection assay.

57. The kit of claim 56, wherein the kit comprises one or more components of a cell free protein expression system and/or a reaction buffer.


SEQUENCE LISTING (46 sequences)

SEQ ID NO: 1 - RNA, 88 nt, synthetic construct
  source 1..88: mol_type = other RNA
gggnnnnnnnnnnnnnnnnnnnnnnnnnnnnnnaacagaggagannnnnnatgnnnnnnnnnaacggtagcgcaggtagcggcatatg

SEQ ID NO: 2 - RNA, 59 nt, synthetic construct
  source 1..59: mol_type = other RNA
catagcatcaatgagtctcagtgaatactgaacagaggagacagtatatgctgagactc

SEQ ID NO: 3 - RNA, 59 nt, synthetic construct
  source 1..59: mol_type = other RNA
ggccttctgtaaaacacgcacagaattttgaacagaggagacaaaatatggtgcgtgtt

SEQ ID NO: 4 - RNA, 59 nt, synthetic construct
  source 1..59: mol_type = other RNA
aattccatctagtattgttatagcggccttaacagaggagaaaggccatgataacaata

SEQ ID NO: 5 - RNA, 59 nt, synthetic construct
  source 1..59: mol_type = other RNA
atactgtgaaattccatctagtattgttataacagaggagaataacaatgctagatgga

SEQ ID NO: 6 - RNA, 59 nt, synthetic construct
  source 1..59: mol_type = other RNA
gtattgttatagcggccttctgtaaaacacaacagaggagagtgtttatgagaaggccg

SEQ ID NO: 7 - RNA, 59 nt, synthetic construct
  source 1..59: mol_type = other RNA
aacacgagcagcctctgatgcaaatgcataaacagaggagatatgcaatggcatcagag

SEQ ID NO: 8 - RNA, 59 nt, synthetic construct
  source 1..59: mol_type = other RNA
cggccttctgtaaaacacgcacagaattttaacagaggagaaaaattatgtgcgtgttt

SEQ ID NO: 9 - RNA, 59 nt, synthetic construct
  source 1..59: mol_type = other RNA
gcagtttcaagagtgcgggagaaaattgataacagaggagaatcaatatgctcccgcac

SEQ ID NO: 10 - RNA, 59 nt, synthetic construct
  source 1..59: mol_type = other RNA
agcagtttcaagagtgcgggagaaaattgaaacagaggagatcaattatgtcccgcact

SEQ ID NO: 11 - RNA, 59 nt, synthetic construct
  source 1..59: mol_type = other RNA
aacacgcacagaattttgagcagtttcaagaacagaggagacttgaaatggctcaaaat

SEQ ID NO: 12 - RNA, 59 nt, synthetic construct
  source 1..59: mol_type = other RNA
ttgagcagtttcaagagtgcgggagaaaataacagaggagaattttcatgcgcactctt

SEQ ID NO: 13 - RNA, 59 nt, synthetic construct
  source 1..59: mol_type = other RNA
attttgagcagtttcaagagtgcgggagaaaacagaggagattctccatgactcttgaa

SEQ ID NO: 14 - RNA, 59 nt, synthetic construct
  source 1..59: mol_type = other RNA
aattttgagcagtttcaagagtgcgggagaaaataacagaggagaattttcatgcgcac

SEQ ID NO: 15 - RNA, 59 nt, synthetic construct
  source 1..59: mol_type = other RNA
cgcacagaattttgagcagtttcaagagtgaacagaggagacactctatgaactgctca

SEQ ID NO: 16 - RNA, 59 nt, synthetic construct
  source 1..59: mol_type = other RNA
acacgcacagaattttgagcagtttcaagaaacagaggagatcttgaatgtgctcaaaa

SEQ ID NO: 17 - RNA, 59 nt, synthetic construct
  source 1..59: mol_type = other RNA
agaaaattgatcgtacaacacgagcagcctaacagaggagaaggctgatggtgttgtac

SEQ ID NO: 18 - RNA, 59 nt, synthetic construct
  source 1..59: mol_type = other RNA
ggagaaaattgatcgtacaacacgagcagcaacagaggagagctgctatggttgtacga

SEQ ID NO: 19 - RNA, 59 nt, synthetic construct
  source 1..59: mol_type = other RNA
ttgttatagcggccttctgtaaaacacgcaaacagaggagatgcgtgatgtacagaagg

SEQ ID NO: 20 - RNA, 75 nt, synthetic construct
  source 1..75: mol_type = other RNA
tttcgctctattctcatcagtttcatgtcctgtgtcggactttagaacagaggagataaagatggacacaggaca

SEQ ID NO: 21 - RNA, 74 nt, synthetic construct
  source 1..74: mol_type = other RNA
tgggatcaagtacatgtagtgcgccacgagcaaaaggactttagaacagaggagataaagatgttttgctcgtg

SEQ ID NO: 22 - RNA, 30 nt, synthetic construct
  source 1..30: mol_type = other RNA
cagtattcactgagactcattgatgctatg

SEQ ID NO: 23 - RNA, 30 nt, synthetic construct
  source 1..30: mol_type = other RNA
caaaattctgtgcgtgttttacagaaggcc

SEQ ID NO: 24 - RNA, 30 nt, synthetic construct
  source 1..30: mol_type = other RNA
aaggccgctataacaatactagatggaatt

SEQ ID NO: 25 - RNA, 30 nt, synthetic construct
  source 1..30: mol_type = other RNA
ataacaatactagatggaatttcacagtat

SEQ ID NO: 26 - RNA, 30 nt, synthetic construct
  source 1..30: mol_type = other RNA
gtgttttacagaaggccgctataacaatac

SEQ ID NO: 27 - RNA, 29 nt, synthetic construct
  source 1..29: mol_type = other RNA
tatgcatttgcatcagaggctgctcgtgt

SEQ ID NO: 28 - RNA, 30 nt, synthetic construct
  source 1..30: mol_type = other RNA
aaaattctgtgcgtgttttacagaaggccg

SEQ ID NO: 29 - RNA, 30 nt, synthetic construct
  source 1..30: mol_type = other RNA
atcaattttctcccgcactcttgaaactgc

SEQ ID NO: 30 - RNA, 30 nt, synthetic construct
  source 1..30: mol_type = other RNA
tcaattttctcccgcactcttgaaactgct

SEQ ID NO: 31 - RNA, 30 nt, synthetic construct
  source 1..30: mol_type = other RNA
cttgaaactgctcaaaattctgtgcgtgtt

SEQ ID NO: 32 - RNA, 30 nt, synthetic construct
  source 1..30: mol_type = other RNA
attttctcccgcactcttgaaactgctcaa

SEQ ID NO: 33 - RNA, 30 nt, synthetic construct
  source 1..30: mol_type = other RNA
ttctcccgcactcttgaaactgctcaaaat

SEQ ID NO: 34 - RNA, 34 nt, synthetic construct
  source 1..34: mol_type = other RNA
attttctcccgcactcttgaaactgctcaaaatt

SEQ ID NO: 35 - RNA, 30 nt, synthetic construct
  source 1..30: mol_type = other RNA
cactcttgaaactgctcaaaattctgtgcg

SEQ ID NO: 36 - RNA, 30 nt, synthetic construct
  source 1..30: mol_type = other RNA
tcttgaaactgctcaaaattctgtgcgtgt

SEQ ID NO: 37 - RNA, 30 nt, synthetic construct
  source 1..30: mol_type = other RNA
aggctgctcgtgttgtacgatcaattttct

SEQ ID NO: 38 - RNA, 30 nt, synthetic construct
  source 1..30: mol_type = other RNA
gctgctcgtgttgtacgatcaattttctcc

SEQ ID NO: 39 - RNA, 30 nt, synthetic construct
  source 1..30: mol_type = other RNA
tgcgtgttttacagaaggccgctataacaa

SEQ ID NO: 40 - RNA, 36 nt, synthetic construct
  source 1..36: mol_type = other RNA
gacacaggacatgaaactgatgagaatagagcgaaa

SEQ ID NO: 41 - RNA, 35 nt, synthetic construct
  source 1..35: mol_type = other RNA
ttttgctcgtggcgcactacatgtacttgatccca

SEQ ID NO: 42 - protein, 7 aa, synthetic construct
ENLYFQG

SEQ ID NO: 43 - DNA, 3636 bp, synthetic construct
  source 1..3636: mol_type = other DNA
agttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattctaatacgactcactatagggtctagaaataattttgtttaactttaagaaggagatatacatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagtaatgaggatcccgggaattctcgagtaaggttaacctgcaggaggcctttaattaaggtggtgcggccgcgctagcggtcccgggggatcgatccggctgctaacaaagcccgaaaggaagctgagttggctgctgccaccgctgagcaataactagcataaccccttggggcctctaaacgggtcttgaggggttttttgctgaaaggaggaactatatccggaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctc

SEQ ID NO: 44 - DNA, 2796 bp, synthetic construct
  source 1..2796: mol_type = other DNA
agttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattctaatacgactcactatagggtctagagggacatacggactcacgtgtccgtatgtcaaatcaattttctcccgcactcttgaaactgcactaaccccttggggcctctaaacgggtcttgaggggttttttgctgaaaggaggaactatatccggaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctc

SEQ ID NO: 45 - DNA, 3804 bp, synthetic construct
  source 1..3804: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcattaatacgactcactatagggtctagaggggcagtttcaagagtgcgggagaaaattgataacagaggagaatcaatatgctcccgcacaacggtagcgcaggtagcggtcatatggaacaaaagttgatttctgaagaagacctgggagagtcattattcaagggtccacgcgactataatcctatctcgtctacaatctgtcatctgactaacgaaagcgatggacatactacgagcctttacggtattggcttcgggccctttattattactaataagcacctgttccgtcgtaataatggaacactgcttgtccagtcgttgcatggcgtattcaaggtgaagaatacgaccacacttcaacagcaccttatcgacggtcgcgatatgatcatcattcgcatgccgaaagatttcccaccatttccgcagaaacttaagttccgcgaaccacagcgtgaggagcgcatctgcctggtgacaacaaactttcagacaaagtcgatgtcaagtatggtctcggatacgagctgtacttttccctcttcggatggcattttttggaagcactggattcagacgaaggacggccaatgtggttcgcctttggtcagtactcgtgacggatttattgtgggaatccatagcgcatcgaactttacaaacactaataactatttcacctctgtacctaaaaactttatggaactgttgactaatcaggaggctcagcaatgggtcagtggctggcgtcttaacgctgatagcgtattgtggggcgggcacaaggtatttatgtccaaacccgaggagccgttccaaccagtgaaagaagcgacacaactgatgtcggagttggtatattcccaatacccatacgatgtcccagactatgcgtaatgaggatcccgggaattctcgagtaaggttaacctgcaggaggcctttaattaaggtggtgcggccgcgctagcggtcccgggggatcgatccggctgctaacaaagcccgaaaggaagctgagttggctgctgccaccgctgagcaataactagcataaccccttggggcctctaaacgggtcttgaggggttttttgatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 46 - DNA, 9563 bp, synthetic construct
  source 1..9563: mol_type = other DNA
ttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatattaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaataattcgagctcggtacctcgcgaatgcatctagatatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccgg